SEQUENCE LISTING (33 sequences)

SEQ ID NO: 1 - DNA, 1503 bp, Mycobacterium smegmatis
  CDS (1)..(1503)
atgagagccctggtagtcatccgcctgtcccgcgtcaccgatgctacgacttcaccggagcgtcagctggagtcttgccagcagctctgcgcccagcgcggctgggacgtcgtcggggtagcggaggatctggacgtctccggggcggtcgatccgttcgaccggaagcgcagaccgaacctggcccggtggctagcgttcgaggagcaaccgttcgacgtgatcgtggcgtaccgggtagaccggttgacccgatcgatccggcatctgcagcagctggtccactgggccgaggaccacaagaagctggtcgtctccgcgaccgaagcgcacttcgatacgacgacgccgtttgcggcggtcgtcatcgcgcttatgggaacggtggcgcagatggaattagaagcgatcaaagagcggaaccgttcggctgcgcatttcaatatccgcgccgggaaataccgaggatccctgccgccgtggggatacctgcctacgcgcgtggacggggagtggcggctggtgccggaccctgtgcagcgagagcgcatcctcgaggtgtatcaccgcgtcgtcgacaaccacgagccgctgcacctggtggcccacgacctgaaccggcgtggtgtcctgtcgccgaaggactacttcgcgcagctgcaaggccgcgagccgcagggccgggagtggtcggctaccgcgctgaagcgatcgatgatctccgaggcgatgctcgggtacgcgactctgaacggtaagaccgtccgagacgacgacggagccccgctggtgcgggctgagccgatcctgacccgtgagcagctggaggcgctgcgcgccgagctcgtgaagacctcccgggcgaagcccgcggtgtctaccccgtcgctgctgctgcgggtgttgttctgcgcggtgtgcggggagcccgcgtacaagttcgccgggggaggacgtaagcacccgcgctaccgctgccgctcgatggggttcccgaagcactgcgggaacggcacggtggcgatggccgagtgggacgcgttctgcgaggagcaggtgctggatctgctcggggacgcggagcgtctggagaaagtctgggtagccggctcggactccgcggtcgaactcgcggaggtgaacgcggagctggtggacctgacgtcgctgatcggctccccggcctaccgggccggctctccgcagcgagaagcactggatgcccgtattgcggcgctggccgcgcggcaagaggagctggagggtctagaggctcgcccgtctggctgggagtggcgcgagaccgggcagcggttcggggactggtggcgggagcaggacaccgcggcaaagaacacctggcttcggtcgatgaacgttcggctgacgttcgacgtccgcggcgggctgactcgcacgatcgacttcggggatctgcaggagtacgagcagcatctcaggctcggcagcgtggtcgaacggctacacaccgggatgtcgtag

SEQ ID NO: 2 - protein, 500 aa, Mycobacterium smegmatis [sequence omitted]

SEQ ID NO: 3 - DNA, 567 bp, Acinetobacter calcoaceticus
  CDS (1)..(567)
atgaaaggccaaaaagtagggtatgtgcgagtgagttcggtcgagcaaaatacagggcgtcaacttgagggaattgaagtcgaccggatttttgttgaccgtgcttcgggtaaaaataccgaccgaccgaaatttcaagaaatgttgaactatgtccgggaaggggatagggtgattgtacattccatggatcgttttgcacgaagtctaaaagatttggtcactgaagtagataaactggtcaaaagagggatcgccatccagtttgtaaaagagaatattacttttactgcccaatccacgccgatggataatttgatgctgcaactgatgggtgcttttgctcaattcgagcgggaaattatcttagaacgtcagaaagaagggattaagatcgcagcagctcagggcaaatataaaggtcgagtccataaattgaatccagatcaagctaaagcattactgcaagcctggaaagaagggaagtattcatcaaaagttgatctggcaaaagcgtttggtatcagtagacaggctgtctatcggtatttaaaacaaattaattag

SEQ ID NO: 4 - protein, 188 aa, Acinetobacter calcoaceticus
MKGQKVGYVRVSSVEQNTGRQLEGIEVDRIFVDRASGKNTDRPKFQEMLNYVREGDRVIVHSMDRFARSLKDLVTEVDKLVKRGIAIQFVKENITFTAQSTPMDNLMLQLMGAFAQFEREIILERQKEGIKIAAAQGKYKGRVHKLNPDQAKALLQAWKEGKYSSKVDLAKAFGISRQAVYRYLKQIN

SEQ ID NO: 5 - DNA, 669 bp, Escherichia coli
  CDS (1)..(669)
atggcgacgcgagagcaacaaccggagggacgaaggttgaaggtcgcccgcatctacctgcgcgccagtacggacgagcagaatcttgaacgccaggagagccttgtagcggccacgcgggccgccgggtactacgtcgccggcatctaccgcgagaaggcgtccggcgcacgcgccgaccggcccgagctgctgcgcatgatcgcggacctgcaacctggtgaagtcgtcgttgcggagaagatcgaccgcatcagccgcttgccgttggccgaggccgagcgcctggttgcgtcgatccgggccaaaggggccaagctggccgtgcctggcgtggtggacctgtcggagctggccgccgaggcgaacggagtggcgaaaatcgttctggaatccgtccaggacatgcttttgaagctcgccttgcagatggcccgcgacgactacgaggatcggcgcgagcgtcaacgtcagggtgtccagttggcgaaggccgccggccgctacaccggccgcaaacgtgacgccggcatgcacgaccgcatcatcgcgcttcgctccggcggatcgagcattgccaagacggccaagctggtcggatgcagcccgagccaggtcaaacgagtgtgggcggcctggaacgcgcagcagcaaaataaaagggcttag

SEQ ID NO: 6 - protein, 222 aa, Escherichia coli
MATREQQPEGRRLKVARIYLRASTDEQNLERQESLVAATRAAGYYVAGIYREKASGARADRPELLRMIADLQPGEVVVAEKIDRISRLPLAEAERLVASIRAKGAKLAVPGVVDLSELAAEANGVAKIVLESVQDMLLKLALQMARDDYEDRRERQRQGVQLAKAAGRYTGRKRDAGMHDRIIALRSGGSSIAKTAKLVGCSPSQVKRVWAAWNAQQQNKRA

SEQ ID NO: 7 - DNA, 561 bp, Shigella flexneri
  CDS (1)..(561)
atgactggacagcgcattgggtatatcagggtcagcaccttcgaccagaacccggaacggcaactggaaggcgtcaaggttgatcgcgcttttagcgacaaggcatccggcaaggatgtcaagcgtccgcaactggaagcgctgataagcttcgcccgcaccggcgacaccgtggtggtgcatagcatggatcgcctggcgcgcaatctcgatgatttgcgccggatcgtgcaaacgctgacacaacgcggcgtgcatatcgaattcgtcaaggaacacctcagttttactggcgaagactctccgatggcgaacctgatgctctcggtgatgggcgcgttcgccgagttcgagcgcgccctgatccgcgagcgtcagcgcgagggtattgcgctcgccaagcaacgcggggcttaccgtggcaggaagaaatccctgtcgtctgagcgtattgccgaactgcgccaacgtgtcgaggctggcgagcaaaagaccaagcttgctcgtgaattcggaatcagtcgcgaaaccctgtatcaatacttgagaacggatcagtaa

SEQ ID NO: 8 - protein, 186 aa, Shigella flexneri
MTGQRIGYIRVSTFDQNPERQLEGVKVDRAFSDKASGKDVKRPQLEALISFARTGDTVVVHSMDRLARNLDDLRRIVQTLTQRGVHIEFVKEHLSFTGEDSPMANLMLSVMGAFAEFERALIRERQREGIALAKQRGAYRGRKKSLSSERIAELRQRVEAGEQKTKLAREFGISRETLYQYLRTDQ

SEQ ID NO: 9 - DNA, 615 bp, Escherichia coli
  CDS (1)..(615)
atgctgatcggctacatgcgggtatcgaaggcggacggatcccagtccaccaatttgcaacgcgatgcgctcatcgctgccggtgtgagccttgcgcacctttacgaggatctggcctcgggcaggcgcgatgatcgcccagggttggctgcttgcctgaaggcgcttcgtgaaggggacacgctagtcgtgtggaagctgcatcggcttggccgtgatctgcgccacctgatcaacaccgtgcacgacctaactgcgcgtagcgtgggcctgaaggtcctgaccggtcacggtgcggcggtcgacacgacgactgccgccggcaagcttgtgttcggtatttttgccgcgctggccgagttcgagcgtgagttgatttccgagcgaacagtcgctggacttatctcggcgcgcgctcgcggcaggaaaggggggcgccccttcaagatgaccgccgccaagctacgcctggcgatggccagcatggggcaaccggaaaccaaggtgggcgatctctgcgaagaactcgggattacccggcagacgctctaccggcacgtgtcgcccaagggcgaactgcggccagacggcgtaaagctgctctccctcggttca

SEQ ID NO: 10 - protein, 205 aa, Escherichia coli
MLIGYMRVSKADGSQSTNLQRDALIAAGVSLAHLYEDLASGRRDDRPGLAACLKALREGDTLVVWKLHRLGRDLRHLINTVHDLTARSVGLKVLTGHGAAVDTTTAAGKLVFGIFAALAEFERELISERTVAGLISARARGRKGGRPFKMTAAKLRLAMASMGQPETKVGDLCEELGITRQTLYRHVSPKGELRPDGVKLLSLGS

SEQ ID NO: 11 - DNA, 561 bp, Escherichia coli
  CDS (1)..(561)
atgcaggggcaccgcatcggctacgtccgggtcagcagcttcgaccagaacccggaacgccagctggaacagacacaggtgagcaaggtgttcaccgacaaggcatcgggcaaggacacccagcgcccccagctcgaagcgctgctgagcttcgtccgcgaaggcgatacagtggtggtgcacagcatggaccggctggcccgcaacctcgatgacctgcgtcgcttggtacagaagctgactcaacgcggcgtgcgcatcgagttcctgaaggagggcctggtgttcactggcgaggactcgccgatggccaacctgatgctgtcggtgatgggggccttcgctgagttcgagcgcgccctgatccgcgagcggcagcgtgagggcatcaccttggccaagcagcgtggcgcgtaccggggccgcaagaaagccctgtccgatgagcaggctgctaccctgcggcagcgagcgacggccggcgagcccaaggcgcagcttgcccgcgagttcaacatcagccgggaaaccctctaccagtacctccgcacggacgactga

SEQ ID NO: 12 - protein, 186 aa, Escherichia coli
MQGHRIGYVRVSSFDQNPERQLEQTQVSKVFTDKASGKDTQRPQLEALLSFVREGDTVVVHSMDRLARNLDDLRRLVQKLTQRGVRIEFLKEGLVFTGEDSPMANLMLSVMGAFAEFERALIRERQREGITLAKQRGAYRGRKKALSDEQAATLRQRATAGEPKAQLAREFNISRETLYQYLRTDD

SEQ ID NO: 13 - DNA, 561 bp, Escherichia coli
  CDS (1)..(561)
atgcaggggcaccgcatcggctacgttcgggtcagcagctttgaccagaacccggaacgccagctggaacaaacccaggtgagcaaggtgttcaccgacaaggcatcgggcaaggacacccagcgcccccagctcgaagcgctgctgagcttcgtccgcgaaggcgatacagtggtggtgcacagcatggatcggctggcccgcaacctcgatgacctgcgtcgcttggtacagaagctgactcagcgcggcgtgcgcatcgagttcctgaaggagggcctggtgttcactggcgaggactcgccgatggccaacctgatgctgtcggtgatgggggccttcgctgagttcgagcgcgccctgatccgcgagcggcagcgtgagggcatcgccttggccaagcagcgtggcgcgtaccggggccgcaagaaagccctgtccgatgagcaggctgctaccctgcggcagcgagcgacggccggcgagcccaaggcgcagcttgcccgcgagttcaacatcagccgggaaaccctctaccagtacctccgcacggacgactga

SEQ ID NO: 14 - protein, 186 aa, Escherichia coli
MQGHRIGYVRVSSFDQNPERQLEQTQVSKVFTDKASGKDTQRPQLEALLSFVREGDTVVVHSMDRLARNLDDLRRLVQKLTQRGVRIEFLKEGLVFTGEDSPMANLMLSVMGAFAEFERALIRERQREGIALAKQRGAYRGRKKALSDEQAATLRQRATAGEPKAQLAREFNISRETLYQYLRTDD

SEQ ID NO: 15 - DNA, 615 bp, Unknown
  Xanthomonas sp. strain W17 - isolated from mercury ores - Kirgizia, former Soviet Union
atgctgataggctacatgcgggtatcgaaggcggacggctcccaggctaccgatttgcagcgcgacgcgctgattgccgccggggtcgatccagtacatctttacgaggaccaggcatccggcatgcgcgaggatcggcccggcttgacgagctgcctgaaggcgttgcgaactggcgacacactggtcgtgtggaaactggatcggctcggacgcgaccagcgacatctcatcaacaccgtgcacgacctgactgggcgcggcatcggcttgaaggtattaaccgggcacggcgcggccatcgacaccacgaccgccgccggcaagctggtctttggcatcttcgccgccctggccgagttcgagcgcgagttgatcgcggagcgcacgattgccggcctagcctcggcccgcgcgcgcgggcggaaaggcggccggccgttcaagatgaccgccgccaagctgcggctggcgatggcggcaatgggtcagccagagaccaaggtcggcgacctgtgccaggaacttggcgtcacgcggcagaccctgtatcggcatgtttcacccaagggtgagctacgtccagatggcgagaagctactcagccgaatttga

SEQ ID NO: 16 - protein, 204 aa, Unknown
  Synthetic Construct
MLIGYMRVSKADGSQATDLQRDALIAAGVDPVHLYEDQASGMREDRPGLTSCLKALRTGDTLVVWKLDRLGRDQRHLINTVHDLTGRGIGLKVLTGHGAAIDTTTAAGKLVFGIFAALAEFERELIAERTIAGLASARARGRKGGRPFKMTAAKLRLAMAAMGQPETKVGDLCQELGVTRQTLYRHVSPKGELRPDGEKLLSRI

SEQ ID NO: 17 - DNA, 1458 bp, Lactococcus lactis
  CDS (1)..(1458)
atgactaagaaagtagcaatctatacacgagtatccactactaaccaagcagaggaagggttctcaattgatgagcaaattgaccgtttaacaaaatatgctgaagcaatggggtggcaagtatctgatacttatactgatgctggtttttcaggggccaaacttgaacgcccagcaatgcaaagattaatcaacgatatcgagaataaagcttttgatacagttcttgtatataagctagaccgcctttcacgtagtgtaagagatactctttatcttgttaaggatgtgttcacaaaaaataaaatagactttatctcgcttaatgaaagtattgatacttcttctgctatgggtagcttgtttctcactattctttctgcaattaatgagtttgaaagagagaatataaaagaacgcatgactatgggtaaactagggcgagcgaaatctggtaagtctatgatgtggactaagacagcttttgggtattaccacaacagaaagacaggtatattagaaattgttcctttacaagctacaatagttgaacaaatattcactgattatttatcaggaatatcacttacaaaattaagagataaactcaatgaatctggacacatcggtaaagatataccgtggtcttatcgtaccctaagacaaacacttgataatccagtttactgtggttatatcaaatttaaggacagcctatttgaaggtatgcacaaaccaattatcccttatgagacttatttaaaagttcaaaaagagctagaagaaagacaacagcagacttatgaaagaaataacaaccctagacctttccaagctaaatatatgctgtcagggatggcaaggtgcggttactgtggagcacctttaaaaattgttcttggccacaaaagaaaagatggaagccgcactatgaaatatcactgtgcaaatagatttcctcgaaaaacaaaaggaattacagtatataatgacaataaaaagtgtgattcaggaacttatgatttaagtaatttagaaaatactgttattgacaacctgattggatttcaagaaaataatgactccttattgaaaattatcaatggcaacaaccaacctattcttgatacttcgtcatttaaaaagcaaatttcacagatcgataaaaaaatacaaaagaactctgatttgtacctaaatgattttatcactatggatgagttgaaagatcgtactgattcccttcaggctgagaaaaagctgcttaaagctaagattagcgaaaataaatttaatgactctactgatgtttttgagttagttaaaactcagttgggctcaattccgattaatgaactatcatatgataataaaaagaaaatcgtcaacaaccttgtatcaaaggttgatgttactgctgataatgtagatatcatatttaaattccaactcgcttaa

SEQ ID NO: 18 - protein, 485 aa, Lactococcus lactis
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 19 - DNA, 1359 bp, Listeria monocytogenes
  CDS (1)..(1359)
atgaaggcagctatttatatacgcgtatctactcaagaacaaatagagaattactctatacaagctcaaactgaaaagctaacagccttgtgccgctcgaaggattgggacgtatacgatattttcatagacggcggatacagcggttcaaacatgaatcgccccgcactaaatgaaatgctaagtaaattacatgaaattgatgctgttgttgtatatcgcttagatagactttcccgctcacaaagagatacgataacgcttattgaagaatacttcttaaaaaacaatgtagaatttgttagtttgtctgaaactcttgacacctctagcccatttgggcgcgcgatgattggtatattatccgtatttgctcaattagagcgcgaaactatacgtgatcgtatggtgatggggaaaattaagcgtattgaagcaggtcttcctttaacgactgcaaaaggtagaacattcggctatgatgttatagatactaaattatatattaatgaagaagaagcaaaacaattacaaatgatttatgatatttttgaggaagaaaaaagcattaccactttacagaagagactaaaaaaattaggattcaaagtgaaatcatatagcagttacaacaattggctaactaatgatttatactgtggttatgtatcttatgcggataaagtgcatacaaaaggtgttcatgagcctattatttcagaggaacaattttatcgagttcaagaaattttttctcgcatgggtaaaaatccaaatatgaatagagattcagcatcgttgctaaataatttggtagtgtgtggaaaatgtgggttgggttttgttcatcggagaaaagatactgtttcccgcggaaaaaaatatcattatagatattatagttgcaagacttacaaacatactcatgaactagaaaaatgtggaaataaaatttggagagctgacaaactcgaggaattaattattgatcgcgtgaataactatagtttcgcttctaggaatgtagataaagaagacgaattagatagcttaaatgaaaaacttaaaacagaacacgtaaaaaagaaacggctatttgatttatatatcagcggttcttacgaagtttcagaacttgatgctatgatggctgatatcgatgctcaaattaattattatgaagcacaaatagaagctaacgaagaattgaagaaaaataaaaagatacaagaaaatttagctgatttagcaacagttgattttgactctttagagttccgagaaaagcaactttatttaaaatcactaattaataaaatttatattgacggtgaacaagttactattgaatggctctag

SEQ ID NO: 20 - protein, 452 aa, Listeria monocytogenes
MKAAIYIRVSTQEQIENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQMIYDIFEEEKSITTLQKRLKKLGFKVKSYSSYNNWLTNDLYCGYVSYADKVHTKGVHEPIISEEQFYRVQEIFSRMGKNPNMNRDSASLLNNLVVCGKCGLGFVHRRKDTVSRGKKYHYRYYSCKTYKHTHELEKCGNKIWRADKLEELIIDRVNNYSFASRNVDKEDELDSLNEKLKTEHVKKKRLFDLYISGSYEVSELDAMMADIDAQINYYEAQIEANEELKKNKKIQENLADLATVDFDSLEFREKQLYLKSLINKIYIDGEQVTIEWL

SEQ ID NO: 21 - DNA, 53 bp, Mycobacterium smegmatis
cagtggtttgtctggtcaaccaccgcggtctcagtggtgtacggtacaaaccc

SEQ ID NO: 22 - DNA, 42 bp, Mycobacterium smegmatis
ccggcttgtcgacgacggcggtctccgtcgtcaggatcatcc

SEQ ID NO: 23 - DNA, 119 bp, Acinetobacter calcoaceticus
cgttactttggggtataccctaaagttacaatataaaagttcttaaaactatgtaacatttaaatgatttttaaccatatataacatgtaactttgatatttaagatttataatttacg

SEQ ID NO: 24 - DNA, 133 bp, Escherichia coli
tcaaattgggtatacccatttgggcctagtctagccggcatggcgcattacagcaatacgcaatttaaatgcgcctagcgcattttcccgaccttaatgcgcctcgcgctgtagcctcacgcccacatatgag

SEQ ID NO: 25 - DNA, 129 bp, Shigella flexneri
aaaatgtcagggaagactctatgaccttcaacgagatatgtcaataaattcaaaattcaatcctatcctgacgcaatttacacatggcatctgacatcaggttagggtatgcctcaacctgacggcggc

SEQ ID NO: 26 - DNA, 189 bp, Escherichia coli
ccgtcacgtatacgttcgattatgtgacagtgtgagccagaaagttctcgccgtcaaaaactgtcacttaacccgtcatttagtctaagacctgcaaacggccattcaggctcgtaatgtgacaaaaactccggcgggatgccctccttggtgaacgtggcgcgcaaacgttccagggattcggggtcg

SEQ ID NO: 27 - DNA, 127 bp, Escherichia coli
ctaccgtcagattgaggcataccctaactggatgtcaggcagggccgcgccgcttagtcagaatagagtcatctttcgcatttttgacacatgcctgcgaaggtcatagatttcagcctgacagaaa

SEQ ID NO: 28 - DNA, 120 bp, Escherichia coli
tgtcaatctaggctataccctaacttgatgtcaggcagggccgcgccgcttcgtcagaatagagtctgctttcccattttttgacacatgcccgcgaaggttatagatttcagcctgaca

SEQ ID NO: 29 - DNA, 189 bp, unknown
  Xanthomonas sp. strain W17 - isolated from mercury ores - Kirgizia, former Soviet Union
ctgtcacatatacgttcgattatgtgacagtgtgagccagaaagttctcgccgtcaaaaactgtcacttaacccgtcatttagtctaagacctgcaaacggccattcaggctcgtaatgtgacaaaaactccggcgggatgccctccttggtgaacgtggcgcgcaaacgttccagggattcggggtcg

SEQ ID NO: 30 - DNA, 65 bp, Lactococcus lactis
tccaactcgcttaattgcgagtttttatttcgtttatttcaattaaggtaactaaaaaactcctt

SEQ ID NO: 31 - DNA, 59 bp, Lactococcus lactis
atgccaacacaattaacatctcaatcaaggtaaatgctttttgctttttttgcgacgtc

SEQ ID NO: 32 - DNA, 57 bp, Listeria monocytogenes
ttgtttagttcctcgttttctctcgttggacggaaacgaatcgagaaactaaaatta

SEQ ID NO: 33 - DNA, 51 bp, Listeria monocytogenes
aactttttcggatcaagctatgaaggacgcaaagagggaactaaacacttg

(19) United States
(12) Patent Application Publication
(10) Pub. No.: US 2011/0136237 A1
(43) Pub. Date: Jun. 9, 2011

(54) SITE-SPECIFIC RECOMBINATION SYSTEMS FOR USE IN EUKARYOTIC CELLS

(75) Inventors: David W. Ow, Hercules, CA (US); James G Thomson, Albany, CA (US)

(73) Assignee: The United States of America, as represented by the Secretary of Agriculture, Washington, DC (US)

(21) Appl. No.: 12/859,229

(22) Filed: Aug. 18, 2010

Related U.S. Application Data
(63) Continuation of application No. 12/494,772, filed on Jun. 30, 2009.
Application No. 12/494,772 is a continuation of application No. 11/209,388, filed on Aug. 22, 2005, now abandoned.
(60) Provisional application No. 60/604,911, filed on Aug. 26, 2004.

(51) Int. Cl. C12N 15/87

(52) U.S. Cl. 435/462; 435/468; 435/440; 435/471

(57) ABSTRACT

Prokaryotic recombination systems have been adapted to function in eukaryotes in order to achieve one or more of the following: DNA site specific excision, translocation, integration and inversion. These recombination systems are identified as seven members of the small serine resolvase subfamily: CinH, ParA, Tn1721, Tn5053, Tn21, Tn402, and Tn501 and three members of the large serine resolvase subfamily: Bxb1, U153, and TP901-1. These recombination systems represent new tools for the genetic manipulation of eukaryotic genomes.

### CROSS REFERENCE TO RELATED APPLICATIONS

The present application is a continuation of copending U.S. patent application Ser. No. 12/494,772, filed Jun. 30, 2009, which claims priority to U.S. patent application Ser. No. 11/209,388, filed Aug. 22, 2005, now abandoned, which claims priority to U.S. Provisional Patent Application Ser. No. 60/604,911 filed on Aug. 26, 2004. Therefore, the present application claims priority to each of the above-referenced patent applications, and each of the above-referenced patent applications is hereby incorporated by reference herein in its entirety.

### BACKGROUND OF THE INVENTION

1. Field of the Invention

The present invention relates to the manipulation of eukaryotic genomes. In particular, the invention is a novel application of prokaryotic recombination systems to eukaryotes, for use in the site-specific excision, inversion, co-integration or translocation of DNA.

2. Description of the Art

Genomic engineering has become an essential tool in the scientific study of various experimental organisms and is also increasingly used in the process of crop improvement. Plant transgenesis, for example, is the process by which a gene from one plant is transferred to another plant, often resulting in new traits being engineered into plants such as enhanced tolerance to herbicides, improved nutrition profiles, resistance to pathogens and abiotic stress, and the ability to detoxify environmental pollutants. These technical advances in plant gene transfer have operated to expand the number of crop species that can be engineered, as well as the efficiency and precision of the gene transfer process itself.

Behind many of the technical advances achieved in plant gene transfer is the development of site-specific recombination systems that enable the precise manipulation of DNA (Ow, 2002). Site-specific recombination permits the precise deletion, inversion, integration or translocation of DNA sequences.

Site-specific DNA excision, for example, permits the removal of selectable marker genes that otherwise would be incorporated into a plant genome that is undergoing genetic modification (Dale and Ow, 1991). The inability to remove various marker genes, such as those that confer antibiotic-resistance, has been at least one deterrent factor in consumer acceptance of GMOs. DNA excision, therefore, is a particularly useful technology in that it can mitigate or even eliminate the transfer of unwanted gene sequences (Heritage 2004).

Additionally, the removal of a particular marker gene allows for it to be reused during subsequent rounds of gene transfer. In fact, site-specific deletion of marker genes from major crop plants such as corn, rice, wheat, cotton and soybean has been achieved (Gilbertson et al., 2003).

DNA integration is currently being explored for the commercial production of crop varieties. Gene transfer via site-specific integration permits a higher rate of transformants with a precise single-copy of the introduced DNA (Albert et al., 1995). More importantly, studies indicate that this process leads to a higher rate of predictable gene expression (Day et al., 2000; Srivastava and Ow, 2002; Srivastava et al., 2004).

DNA translocation, the process of moving a segment of DNA to a defined locus in another chromosome, is currently in the experimental stage of development. Site-specific DNA translocation could theoretically facilitate the introgression of transgenes from a single laboratory variety used for DNA transformation to a large number of field-grown cultivars (elite lines) in different parts of the world.

Numerous site-specific recombination systems have been described in prokaryotic and lower eukaryotic organisms. Each recombination system consists of a recombinase enzyme and a set of recombination sites. Recombinase binding to the two recombination sites assemble into a synaptonemal complex, which is then followed by precise cleavage and strand exchange that results in a recombination event with neither loss nor gain of genetic material. Based on biochemical properties of the recombination reaction, the recombinase family is divided into two subfamilies, the tyrosine recombinases and the serine recombinases. The distinction between these groups is due to differences in the catalytic site design and the mode of action.

It is also useful to group the recombinases into those that can catalyze reversible reactions (bi-directional), and those that cannot (uni-directional). To date only members of the tyrosine recombinase subfamily has shown bi-directional activity. Those that catalyze uni-directional reactions are members of both the tyrosine and the serine recombinase subfamilies. Furthermore, within the serine recombinase subfamily, there are members dedicated for the deletion reaction but cannot catalyze inversion or integration reactions.

According to the biochemical classification described above, three recombination systems popularly used in plants belong to the tyrosine family. These are Cre-lox, FLP-FRT, R-RS: Cre, FLP and R are the recombinases; and lox, FRT and RS are the respective recombination sites. These systems are similar in that recombination with two substrate sites generates product sites of the same sequence. The recombination reaction is fully reversible. In these reversible recombination systems, when the two participating substrate sites are in direct orientation in cis, in inverted orientation in cis, or in different molecules in trans, the recombination will lead, respectively, to a deletion, inversion, or integration reaction. The reversible nature of these recombination systems, however, is often a hindrance to genetic engineering because an intended event can be reversed. For example, the integration of DNA using a reversible recombination system can result in the DNA being excised again by the reverse reaction.

Another recombination system being used in plants is phiC31, a member of the large serine recombinase subfamily. In this system, a phiC1 integrase acts on two different sequences, attB and attP, in which recombination between these sequences generate hybrid sites known as attL and attR. Unlike the Cre-lox, FLP-FRT and R-RS systems, the phiC31 integrase alone cannot reverse the attB×attP recombination reaction. While phiC31, Cre-lox, FLP-FRT, and R-RS are useful tools for genetic engineering, having additional recombination systems, especially those that catalyze non-reversible reactions, would offer more options for the genetic manipulation of a genome.

### SUMMARY OF THE INVENTION

To provide additional DNA manipulation tools for plant genetic modification, a collection of new prokaryotic recombination systems have been identified for use in eukaryotes. Like the Cre-lox and phiC31 systems, the bacterial recombinases described herein should be suitable for the precise genetic modification of eukaryotic genomes.

The systems have been designated Bxb1, U153, and TP901-1 of the large serine recombinase subfamily; and CinH, ParA, Tn1721, Tn5053, Tn21, Tn402, and Tn501 of the small serine recombinase subfamily.

The CinH, ParA, Tn1721, Tn5053, Tn21, Tn402, and Tn501 systems can cause site-specific deletions, such as for the purpose of removing selectable marker genes or other unneeded DNA from eukaryotic cells, including the removal of nearly all exogenously introduced DNA from a transgene locus. The excision reaction does not reverse, as these systems do not perform integration reactions. Some of these systems can also perform inversions. Of particular significance is that these recombination systems require recombination targets much larger than those of the Cre-lox, the FLP-FRT, or the R-RS system. Unlike the relatively small lox, FRT and RS sites (34 by or less), the recombination sites of these systems range from 100 to 200 bp. The larger-size requirement for target specificity lessen the probability of unintended recombination with native host sequences that may resemble the intended target.

The Bxb1, U153 and TP901-1 systems are capable of performing excision, inversion and co-integration reactions. Moreover, as these recombination reactions are uni-directional, the reverse reaction is prevented. As with the phiC31 system, the Bxb1, U153 and TP901-1 systems are ideally suited for integrating DNA into the host genome through integration into a transgenic recombination site, or a native host sequence that functionally operates as a complementary recombination site, since the integrated molecule will not be re-excised by the integrase protein without an additional excisionase cofactor. In the case of TP901-1, site-specific integration in mammalian cells has recently been shown (Stoll et al., 2002).

### BRIEF DESCRIPTION OF THE DRAWINGS

FIG. 1 is a schematic representation of the generic excision assay. It shows (a) detection construct pPB-X containing an eGFP ORF flanked by complementary recombination sites of recombination system “X”, and where recombination sites are oriented for deletion of eGFP. Depending on the recombination system, the recombination sites, shown as filled or open arrowheads, may or may not be identical in sequence. Example shown are two different sequences, attB and attP. (b) Recombinase is provided by the cointroduced construct pNMT-X, where “X” recombinase is produced by the promoter P_{NMT}. (c) Expected excision products are pPBexc-X, a replicating plasmid maintained by the autonomous replication sequence ARS and facilitated by selection of the leu marker, and a circular eGFP fragment that is replication deficient. Primers, shown as small arrowheads, are expected to amplify a PCR product of ˜1.4 kb or ˜0.74 kb, respectively, before (a) or after (c) site-specific excision of eGFP (depending on the length of the recombination sites, the sizes of these PCR product may differ slightly). Endonuclease sites shown are AatII (At), AscI (A), NheI (Nh), NotI (N), PstI (P), SacI (S). AscI and SacI are expected to cleave a 1.8 kb or 0.96 kb fragment, respectively, before (a) or after (c) site-specific excision of eGFP. Not to scale, gene terminators, and promoters for his, ura4 and leu not shown.

FIG. 2 is a schematic representation of the genomic excision assay. Part (a) shows pRLPB-X containing an eGFP ORF flanked by complementary recombination sites of recombination system “X”, and where recombination sites are oriented for deletion of eGFP. Depending on the recombination system, the recombination sites, shown as filled or open arrowheads, may or may not be identical in sequence. Example shown are two different sequences, attB and attP. The pRLPB-X construct contains the ura4 gene for homologous recombination and selection but lack an ARS for episomal replication. (b) Linear pRLPB-X DNA from Stu1 cleavage for integration into the ura4-294 allele. (c) Structure of target line ura4 locus after homologous insertion of pRLPB-X DNA, with the genomic attB site and attP sites flanking eGFP. (d) Target lines transformed by pNMT-X, which produces recombinase “X” from the promoter P_{NMT}, leading to excision of eGFP as depicted in (e). Primers, shown as small arrowheads (c, e), amplify a PCR product of ˜1.6 kb or ˜0.80 kb, respectively, before or after site-specific excision of eGFP (length of PCR product may vary depending on the length of the recombination sites used). Not to scale, gene terminators, and promoters for bsd, ura4 and leu not shown.

FIG. 3 is a schematic representation of a generic inversion assay. It shows (a) detection construct pPBi-X contains an eGFP ORF flanked by complementary recombination sites of recombination system “X”, and where recombination sites are positioned in the orientation for inversion of eGFP. Depending on the recombination system, the recombination sites, shown as filled or open arrowheads, may or may not be identical in sequence. Example shows two different sequences, attB and attP. (b) Recombinase is provided by the cointroduced construct pNMT-X, where “X” recombinase is produced by the promoter P_{NMT}. Expected inversion of the eGFP fragment within pPBi-X lead to the structure shown in (c). Primers 1 and 3, shown as small arrowheads, are expected to amplify a PCR product of ˜1.6 kb only after site-specific inversion of eGFP. Not to scale, gene terminators, and promoters for his, ura4 and leu not shown.

FIG. 4 shows a schematic representation of intermolecular recombination. It shows (a) Acceptor construct pHisB-X contains a recombination site, a downstream ura4 ORF, a his3 marker for selection and an ARS for autonomous replication. (b) Donor construct pLeuP-X contains P_{NMT}, a downstream complementary recombination site, a leu marker for selection, but devoid of an ARS. Depending on the recombination system, designated here as “X”, the recombination sites, shown as filled or open arrowheads, may or may not be identical in sequence. Example shows two different sequences, attB and attP. (c) Recombinase is provided by pNMTAS-X that lacks a selectable marker and an ARS. (d) PCR primers, shown as small arrowheads, amplify a ˜0.74 kb band upon intermolecular site-specific recombination that fuses P_{NMT} to the ura4 ORF to form pHisLeuX (length of PCR product may vary depending on the length of the recombination sites used). Endonuclease sites shown are PstI (P), AscI (A), NheI (N), SacI (S). Not to scale, gene terminators, and promoters for his, ura4 and leu not shown.

FIG. 5 shows a schematic representation of recombinase-mediated integration of DNA into the S. pombe chromosome. It shows (a) pRLBZ-X (“X” denotes particular recombination system) contains attB with a downstream bsd ORF but devoid of an upstream promoter. (b) Stu1-cleaved pRLBZ-X was inserted into ura.294 allele via homologous recombination to form target lines with structures shown in (c). (d) pLeuP-X and pNMTAS-X were introduced into the target lines and X recombinase-mediated integration of pLeuP-X into the locus generates the structure shown in (e). PCR primers, shown as small arrowheads, generated a ˜0.8 kb band (length of PCR product may vary depending on the length of the recombination sites used), the predicted junction product from insertion of pLeuP-X into the genomic attB site shown in (c). Not to scale, gene terminators, and promoters for bsd, ura4 and leu not shown.

FIG. 6 shows a schematic representation of site-specific recombination in plant cells. It shows (a) Detection construct pN6PB-X contains an eGFP ORF flanked by complementary recombination sites of recombination system “X”, and where recombination sites are oriented for deletion of eGFP. Depending on the recombination system, the recombination sites, shown as filled or open arrowheads, may or may not be identical in sequence. Example shown are two different sequences, attB and attP. (b) Agrobacterium-mediated transformation integrates a copy of pN6PB-X T-DNA into the plant genome. Recombinase is provided through a second transformation with pCK-X shown in (c), leading to transgenic lines doubly transformed with both pN6PB-X and pCK-X (d), after recombinase-catalyzed deletion of eGFP. (e) pN6PB-X and pCK-X DNA transfected into plant protoplasts. Recombinase-catalyzed site-specific recombination produces the deletion plasmid shown. Primers, shown as small arrowheads, amplify a PCR product of ˜1.5 kb or ˜0.50 kb, respectively, before or after site-specific excision of eGFP (length of PCR product may vary depending on the length of the recombination sites used). Not to scale, gene terminators, and promoters for gus (beta-glucuronidase gene), hpt (hygromycin resistance gene) and npt (kanamycin resistance gene) not shown. RB, LB: T-DNA right and left borders, respectively.

FIG. 7 is a schematic representation of site-specific integration in plants. It shows (a) Target site construct pYMP72 comprises of npt, gus, and attP within the T-DNA left (LB) and right (RB) borders of a pCambia2300 vector backbone (Cambia, Canberra). Agrobacterium-mediated transformation yields the structure shown in (b). (c) Transgenic lines further transformed with pYWSB2 with or without pCK-X or pCK-Xn that may lead to the structures shown in (d) or (e), depending on recombination of the genomic attP with either of the two attB sites in pYWSB2. pCK-Xn differs from pCK-X in that the “Xn” recombinase is fused to a nuclear localization signal. Endonuclease site shown is XbaI (X). Not to scale, gene terminators, and promoters for gus (beta-glucuronidase gene), luc (luciferase gene), hpt (hygromycin resistance gene) and npt (kanamycin resistance gene) not shown.

FIG. 8 is a schematic representation of intermolecular recombination in mammalian cells. It shows (a) Acceptor construct pBEIN-X contains an attB recombination site, a downstream eGFP ORF, an IRES for bicistronic translation and the neomycin resistance marker Neo. (b) The donor construct pQCAP-X contains CMV promoter and a downstream attP recombination site but devoid of a selection marker. Depending on the recombination system, designated here as “X”, the recombination sites, shown as filled or open arrowheads, may or may not be identical in sequence. (c) Recombinase is provided by pLIC-X which lacks a selectable marker. (d) Expected recombination product that may express eGFP if the attR site does not interfere with downstream gene expression. HEK293 cells transfected with the 3 constructs were analyzed UV-microscopy and FACS for eGFP expression. Graphic representation of FACS of HEK293 cells transfected with pBEIN-X and pQCAP-X (e) and with the combination of pBEIN-X, pQCAP-X and pLIC-X (f). eGFP expression detected in ˜25% of the cell population. Not to scale, gene terminators, and promoter for Neo (neomycin resistance gene) not shown.

### BRIEF DESCRIPTION OF THE SEQUENCES

SEQ ID NO:1 is the cDNA coding for the Bxb1 recombinase.

SEQ ID NO:2 is the amino acid sequence comprising the Bxb1 recombinase.

SEQ ID NO:3 is the cDNA coding for the CinH enzyme.

SEQ ID NO:4 is the amino acid sequence comprising the CinH recombinase.

SEQ ID NO:5 is the cDNA coding for the ParA enzyme.

SEQ ID NO:6 is the amino acid sequence comprising the ParA recombinase.

SEQ ID NO:7 is the cDNA coding for the Tn21 enzyme.

SEQ ID NO:8 is the amino acid sequence comprising the Tn21 recombinase.

SEQ ID NO:9 is the cDNA coding for the Tn402 enzyme.

SEQ ID NO:10 is the amino acid sequence comprising the Tn402 recombinase.

SEQ ID NO:11 is the cDNA coding for the Tn501 recombinase.

SEQ ID NO:12 is the amino acid sequence comprising the Tn501 recombinase.

SEQ ID NO:13 is the cDNA coding for the Tn1721 recombinase.

SEQ ID NO:14 is the amino acid sequence comprising the Tn1721 recombinase.

SEQ ID NO:15 is the cDNA coding for the Tn5053 recombinase.

SEQ ID NO:16 is the amino acid sequence comprising the Tn5053 recombinase.

SEQ ID NO:17 is the cDNA coding for the TP901 recombinase.

SEQ ID NO:18 is the amino acid sequence comprising the TP901 recombinase.

SEQ ID NO:19 is the cDNA coding for the U153 recombinase.

SEQ ID NO:20 is the amino acid sequence comprising the U153 recombinase.

SEQ ID NO:21 is a DNA attP recombination site for the Bxb1 recombinase.

SEQ ID NO:22 is a DNA attB recombination site for the Bxb1 recombinase.

SEQ ID NO:23 is a DNA recombination site for the CinH recombinase.

SEQ ID NO:24 is a DNA recombination site for the ParA recombinase.

SEQ ID NO:25 is a DNA recombination site for the Tn21 recombinase.

SEQ ID NO:26 is a DNA recombination site for the Tn402 recombinase.

SEQ ID NO:27 is a DNA recombination site for the Tn501 recombinase.

SEQ ID NO:28 is a DNA recombination site for the Tn1721 recombinase.

SEQ ID NO:29 is a DNA recombination site for the Tn5053 recombinase.

SEQ ID NO:30 is a DNA attP recombination site for the Tp901 recombinase.

SEQ ID NO:31 is a DNA attB recombination site for the Tp901 recombinase.

SEQ ID NO:32 is a DNA attP recombination site for the U153 recombinase.

SEQ ID NO:33 is a DNA attB recombination site for the U153 recombinase.

### DEFINITIONS

Unless defined otherwise, all technical and scientific terms used herein have the meaning commonly understood by a person skilled in the art to which this invention belongs. The following references provide one of skill with a general definition of many of the terms used in this invention: Lodish, et al., MOLECULAR CELL BIOLOGY (5th ed. 2004); and Nelson, LEHNINGER PRINCIPLES OF BIOCHEMISTRY (3d ed. 2000). To facilitate understanding of the invention, a number of terms are defined below.

Bxb1 is the abbreviation for the Bxb1 recombination system derived from Mycobacterium smegmati bacteriophage Bxb1, which is associated with the enzyme Bxb1 resolvase (or recombinase), also identified in SEQ ID NO: 2.

U153 is the abbreviation for the U153 recombination system derived from Listeria monocytogenes bacteriophage U153, which is associated with the enzyme U153 integrase (recombinase), also identified in SEQ ID NO: 20.

CinH is the abbreviation for the CinH recombination system derived from Acetinetobacter plasmids pKLH2, pKLH2O4 and pKLH205, which is associated with the enzyme CinH recombinase, also identified in SEQ ID NO: 4.

ParA is the abbreviation for the ParA recombination system derived from plasmids RK2 and RP4 which is associated with the enzyme ParA recombinase, also identified in SEQ ID NO: 6.

Tn1721 recombination system is derived from transposable element Tn1721, which is associated with the enzyme Tn1721 resolvase (recombinase), also identified in SEQ ID NO: 14.

Tn5053 recombination system is derived from transposable element Tn5053, which is associated with the enzyme Tn5053 resolvase (recombinase), also identified in SEQ ID NO: 16.

TP901-1 (also known as TP 901) is the abbreviation for the recombinase system derived from bacteriophage TP901-1 that infects the bacteria Lactococcus lactis subsp. cremoris 901-1, which is associated with the enzyme Tn901-1 integrase also identified in SEQ ID NO: 18.

Tn21 recombination system is derived from transposable element Tn21, which is associated with the enzyme Tn21 resolvase (recombinase), also identified in SEQ ID NO: 8. Tn21 is 80% similar on the nucleotide level and 84.7% on the amino acid level to Tn1721. Testing in bacteria has shown the ability to exchange res site for successful recombination events (Rogowsky and Schmitt, 1985).

Tn402 recombination system is derived from transposable element Tn402, which is associated with the enzyme Tn402 resolvase (recombinase), also identified in SEQ ID NO: 10. Tn402 is 83% similar on the nucleotide level and 86.8% on the amino acid level to Tn5053. Testing in bacteria has shown the ability to exchange res site for successful recombination events (Kholodii, 1995, Kholodii, et. al., 1995).

Tn501 recombination system is derived from transposable element Tn501, which is associated with the enzyme Tn501 resolvase (recombinase), also identified in SEQ ID NO: 12. Tn501 is 99% similar on the nucleotide level and 99.5% on the amino acid level to Tn1721. Testing in bacteria has shown the ability to exchange res site for successful recombination events (Rogowsky and Schmitt, 1985).

“Autotrophy” means capable of growth in the prescribed growth media without supplementation other nutrients.

“Cotransformation” refers to the co-introduction of DNA molecules into the same host cell.

“DNA excision” refers to a manipulation whereby a nucleotide or a sequence of nucleotides has been removed from the DNA. “DNA Deletion” is synonymous with DNA excision.

“DNA translocation” refers to the process of moving a segment of DNA to a defined locus in another chromosome.

“DNA integration” refers to the insertion of DNA into a location in another DNA molecule.

“DNA inversion” refers to the inversion of a sequence of DNA nucleotides.

“Synaptonemal complex” is defined as a structure that holds chromosomes, or strands of DNA together to promote genetic recombination.

“Transfected” means the transient introduction of DNA into a host.

AfeI is an endonuclease.

ARS is autonomous replicating sequence used in DNA molecules that replicate in a host cell.

AscI is an endonuclease.

att refers to a recombination site (attachment site of the recombinase).

attB refers to recombination (attachment) site from bacteria.

attP refers to recombination (attachment) site from bacteriophage.

attL refers to one of two hybrid attachment sites generated by the recombination between attB and attP.

attR refers to one of two hybrid attachment sites generated by the recombination between attB and attP.

BcII is an endonuclease.

EagI is an endonuclease.

eGFP is the coding region for the enhanced green florescence protein eGFP which is used as a visual genetic marker.

Electroporation is a method to introduce DNA into a host cell.

his3 is a yeast gene in the biosynthesis of the amino acid histidine.

Leu⁺His⁺ colonies refer to colonies capable of growth without supplementation of leucine or histidine.

lox means locus of crossover (recombination site of Cre recombinase).

MRS is multimer resolution site, used for example in reference to the recombination site used by the ParA recombination system.

NheI is an endonuclease.

NotI is an endonuclease.

parCBA are three genes that along wth parDE regulate the maintenance of plasmids RK2 and RP4.

pPB-Cre is a molecular DNA construct as defined in the text.

parDE are two genes that along wth parCBA regulate the maintenance of plasmids RK2 and RP4.

pHisB-X is a molecular DNA construct as defined in the text.

pLeuP-X is a molecular DNA construct as defined in the text.

pNMT is a molecular DNA construct as defined in the text.

pNMT-TOPO is a commercially available cloning vector.

P_{NMT} is a promoter whose activity is repressible by thiamine.

pNMT-X is a molecular DNA construct as defined in the text.

pNMTAS-X a molecular DNA construct as defined in the text.

pPB-X is a molecular DNA construct as defined in the text.

pLT43 is a molecular DNA construct as defined in the text.

pRSD1 is conjugative plasmid.

pPBexc-X is a molecular DNA construct as defined in the text.

Recombinase ORFs are coding sequences for recombinase proteins.

res refers to resolution site, often used for the recombination site of recombination systems derived from the resolvase family.

SacI is an endonuclease.

S. pombe FY527 is a strain of Schizosaccharomyces pombe.

StuI is an endonuclease.

SacI is an endonuclease.

ura4 is a S. pombe gene in the biosynthesis of uracil.

The term “transgenic” when used in reference to a cell refers to a cell which contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

The term “transgene” as used herein refers to any nucleic acid sequence that is introduced into the genome of a cell by experimental manipulations. A transgene may be a “native DNA sequence,” or a “heterologous DNA sequence” (i.e., “foreign DNA”). The term “native DNA sequence” refers to a nucleotide sequence which is naturally found in the cell into which it is introduced so long as it does not contain some modification (e.g., a point mutation, the presence of a selectable marker gene, etc.) relative to the naturally-occurring sequence.

The term “heterologous DNA sequence” refers to a nucleotide sequence that is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes a native DNA sequence that contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

The term “transformation” as used herein refers to the introduction of a transgene into a cell. Transformation of a cell may be stable or transient.

The term “stable transformation” or “stably transformed” refers to the introduction and integration of one or more transgenes into the genome of a cell. Stable transformation of a cell may be detected by Southern blot hybridization of genomic DNA of the cell with nucleic acid sequences that are capable of binding to one or more of the transgenes. Stable transformation of a plant may also be detected by using the polymerase chain reaction to amplify transgene sequences from genomic DNA from cells of the progeny of that plant. The term “stable transformant” refers to a cell that has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene.

The term “isolated” when used in relation to a nucleic acid molecule, as in “an isolated nucleic acid sequence” refers to a nucleic acid sequence that is identified and separated from at least one contaminant nucleic acid with which it is ordinarily associated in its natural source. Isolated nucleic acid is nucleic acid present in a form or setting that is different from that in which it is found in nature. The isolated nucleic acid sequence may be present in single-stranded or double-stranded form. When an isolated nucleic acid sequence is to be utilised to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain of Fusarium, or another or related organism and thus, for example, may be an allelic or species variant of the polypeptide encoding region of the nucleic acid sequence.

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least about 20% pure, preferably at least about 40% pure, more preferably at least about 60% pure, even more preferably at least about 80% pure, and most preferably at least about 90% or 95% pure. Purity may be determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

The term “identity,” as known in the art, is a relationship between two or more polypeptide sequences or two or more polynucleotide sequences, as determined by a comparison of the sequences. In the art, “identity” also means the degree of sequence relatedness between polypeptide or polynucleotide sequences, as determined by the match between strings of such sequences. “Identity” can be readily calculated by known methods, including but not limited to those described in Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; and Sequence Analysis in Molecular Biology, von Heinje, G., Academic Press, 1987. Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990) and Altschul et al., Gapped BLAST and PSI-BLAST: a new generation of protein database search programs. Nucleic Acids Res. 25: 3389-3402 (1997).), ALIGN (http://dot.imgen.bcm.tmc.edu:933 1/seq-search/alignment.html), and ClustalW (http://dot.imgen.bcm.edu:9331/cgi-bin/multi-align/multi-align.p1) (Higgens, 1989).

“Percentage of sequence identity” is determined by comparing two optimally aligned sequences over a comparison window, wherein the portion of the polynucleotide sequence or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. It is preferred that the comparison window is at least 50% of the coding sequence, preferably 60%, more preferably 75% or 85%, and even more preferably 95% to 100%.

The term “hybridization” as used herein includes “any process by which a strand of nucleic acid joins with a complementary strand through base pairing.”[Coombs J (1994) Dictionary of Biotechnology, Stockton Press, New York N.Y.]. Hybridization and the strength of hybridization (i.e., the strength of the association between the nucleic acids) is impacted by such factors as the degree of complementarity between the nucleic acids, stringency of the conditions involved, the T.sub.m of the formed hybrid, and the G:C ratio within the nucleic acids.

An “exogenous DNA segment”, “heterologous polynucleotide” a “transgene” or a “heterologous nucleic acid”, as used herein, is one that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

The term “gene” is used broadly to refer to any segment of DNA associated with a biological function. Thus, genes include coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “isolated”, when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

The term “naturally-occurring” is used to describe an object that can be found in nature as distinct from being artificially produced by man. For example, a polypeptide or polynucleotide sequence that is present in an organism (including viruses) that can be isolated from a source in nature and which has not been intentionally modified by man in the laboratory is naturally-occurring.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzeretal. (1991) Nucleic Acid Res. 19: 5081; Ohtsukaetal. (1985) J. Biol. Chem. 260: 2605-2608; Cassol et al. (1992); Rossolini et al. (1994) Mol. Cell. Probes 8: 91-98). The term nucleic acid is used interchangeably with gene, cDNA, and mRNA encoded by a gene.

“Nucleic acid derived from a gene” refers to a nucleic acid for whose synthesis a gene, or a subsequence thereof (e.g., coding region), has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative 30 of the presence and/or abundance of the original.

A DNA segment is “operably linked” when placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers, for example, need not be contiguous with the coding sequences whose transcription they control. Linking is accomplished by ligation at convenient restriction sites or at adapters or linkers inserted in lieu thereof

“Plant” includes whole plants, plant organs (e.g., leaves, stems, roots, etc.), seeds and plant cells and progeny of same. The class of plants that can be used in the methods of the invention is generally as broad as the class of higher plants amenable to transformation techniques, including both monocotyledonous and dicotyledonous plants.

“Promoter” refers to a region of DNA involved in binding the RNA polymerase to initiate transcription. An “inducible promoter” refers to a promoter that directs expression of a gene where the level of expression is alterable by environmental or developmental factors such as, for example, temperature, pH, transcription factors and chemicals.

The term “recombinant” when used with reference to a cell indicates that the cell replicates a heterologous nucleic acid, or expresses a peptide or protein encoded by a heterologous nucleic acid. Recombinant cells can contain polynucleotides that are not found within the native (non-recombinant) form of the cell. Recombinant cells can also contain polynucleotides found in the native form of the cell wherein the polynucleotides are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

“Recombinase” refers to an enzyme that catalyzes recombination between two or more recombination sites. Recombinases useful in the present invention catalyze recombination at specific recombination sites that are specific polynucleotide sequences that are recognized by a particular recombinase. The term “integrase” or “resolvase” refers to types of recombinase.

“Transformation rate” refers to the percent of cells that successfully incorporate a heterologous polynucleotide into its genome and survive.

The term “transgenic” refers to a cell that includes a specific modification that was introduced into the cell, or into an ancestor of the cell. Such modifications can include one or more point mutations, deletions, insertions, or combinations thereof. When referring to an animal, the term “transgenic” means that the animal includes cells that are transgenic. An animal that is composed of both transgenic and non-transgenic cells is referred to herein as a “chimeric” animal.

The term “vector” refers to a composition for transferring a nucleic acid (or nucleic acids) to a host cell. A vector comprises a nucleic acid encoding the nucleic acid to be transferred, and optionally comprises a viral capsid or other materials for facilitating entry of the nucleic acid into the host cell and/or replication of the vector in the host cell (e.g., reverse transcriptase or other enzymes which are packaged within the capsid, or as part of the capsid).

“Recombination sites” are specific polynucleotide sequences that are recognized by the recombinase enzymes described herein. Recombination occurs at two recombination sites that participate in the recombination reaction. Each of the two participating sites may comprise of identical or near identical sequences, or may comprise of non-identical sequences or sequences with low sequence similarity or homology. The two participating sites may be present in the same molecule, or present in different molecules. For recombination involving the recombination of two non-identical sites, they are also termed “complementary sites.” For example one complementary site is present in the target nucleic acid (e.g., a chromosome or episome of a eukaryote) and another on the nucleic acid that is to be integrated at the target recombination site. The terms “attB” and “attP” which refer to attachment (or recombination) sites originally from a bacterial chromosome and a phage chromosome, respectively, are used herein although recombination sites for particular enzymes may have different names. The recombination sites typically include left and right arms separated by a core or spacer region. Thus, an attB recombination site consists of BOB', where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “attR”. The attL and attR sites, using the terminology above, thus consist of BOP′ and POB′, respectively. In some representations herein, the “O” is omitted and attB and attP, for example, are designated as BB′ and PP′, respectively.

“Site specific” refers to, but is not limited to, recombination or a recombination event which occurs at a predictable locus or identifiable nucleotide sequence.

“Recombination” refers to the process in which DNA molecules are broken and the fragments rejoined in new combinations. The process may include the deletion or excision of some fragments from the resulting product.

“Recombination systems” include particular recombinases and their associated recombination sites. For example, the Bxb1 recombination system includes the nucleotide disclosed in SEQ ID NO: 1, the protein disclosed in SEQ ID NO: 2, and the attachment sites disclosed in SEQ ID NO: 21 and SEQ ID NO: 22. Promoters may or may not be included.

### DETAILED DESCRIPTION OF THE INVENTION

The present invention provides methods for obtaining site-specific recombination in eukaryotic cells. Unlike previously known systems for obtaining site-specific recombination in eukaryotes, these recombination systems use different recombination proteins (recombinases) and different recombination sites.

The methods involve contacting a pair of recombination sites (e.g., attB and attP) that are present in a eukaryotic cell with a corresponding recombinase. The recombinase then mediates recombination between the recombination sites. Depending upon the relative locations of the two recombination sites, any one of a number of events can occur as a result of the recombination. For example, if the two recombination sites are present on different nucleic acid molecules, the recombination can result in integration of one nucleic acid molecule into a second molecule. Thus, one can obtain integration of a plasmid that contains one recombination site into a eukaryotic cell chromosome that includes the corresponding recombination site.

The two recombination sites can also be present on the same nucleic acid molecule. In such cases, the resulting product depends upon the relative orientation of the sites. For example, for recombination sites are that identical or nearly identical in sequence, recombination between sites that are in the direct orientation will result in excision of any DNA that lies between the two recombination sites. In contrast, recombination between sites that are in the reverse orientation can result in inversion of the intervening DNA. One example of an application for which this method is useful involves the placement of a promoter between the two recombination sites. If the promoter is initially in the opposite orientation relative to a coding sequence that is to be expressed by the promoter and the recombination sites that flank the promoter are in the inverted orientation, contacting the recombination sites will result in inversion of the promoter, thus placing the promoter in the correct orientation to drive expression of the coding sequence. Similarly, if the promoter is initially in the correct orientation for expression and the recombination sites are in the same orientation, contacting the recombination sites with the promoter can result in excision of the promoter fragment, thus stopping expression of the coding sequence.

The methods of the invention are also useful for obtaining translocations of chromosomes. In these embodiments, one recombination site is placed on one chromosome and a second recombination site that can serve as a substrate for recombination with the first recombination site is placed on a second chromosome. Upon contacting the two recombination sites with a recombinase, recombination occurs that results in swapping of the two chromosome arms. For example, one can construct two strains of an organism, one strain of which includes the first recombination site and the second strain that contains the second recombination site. The two strains are then crossed, to obtain a progeny strain that includes both of the recombination sites. Upon contacting the sites with the recombinase, chromosome arm swapping occurs.

Moreover, the technique and recombination systems introduced here can overcome the inherent problem of random DNA insertion by Agrobacterium-mediated site-specific integration. This can be achieved by using two recombinases to drive integration. In some of the disclosed systems, the first recombinase circularizes a portion of the DNA from the linear Agrobacterium T-DNA intermediate via excision that prevents reintroduction of the modified DNA into the parent construct. The second recombinase integrates the excised circular DNA into a genomic site that has a complementary recombinase site recognized by the second recombinase. This process is also accomplished in a uni-directional manner; the integrating recombinase is incapable of excising the introduced DNA. The systems described here offer the option of using two uni-directional recombinases for site-specific integration of DNA, which has greater control over the use of a single bi-directional recombination system.

### Recombinases and Recombination Sites

The methods of the invention use recombinase systems to achieve integration or other rearrangement of nucleic acids in eukaryotic cells. A recombinase system typically consists of three elements: two specific DNA sequences (“the recombination sites”) and a specific enzyme (“the recombinase”). The recombinase catalyzes a recombination reaction between the specific recombination sites.

Recombination sites have an orientation. In other words, they are not palindromes. The orientation of the recombination sites in relation to each other determines what recombination event takes place. When present in the same DNA molecule, the recombination sites may be in two different orientations: direct (same orientation) or indirect (opposite orientation). When the recombination sites are present on a single nucleic acid molecule and are in a direct orientation to each other, then the recombination event catalyzed by the recombinase is an excision of the intervening nucleic acid, leaving a single recombination site. When the recombination sites are in the opposite orientation, then the intervening sequence is inverted.

The recombinases used in the methods of the invention can mediate site-specific recombination between a first recombination site and a second recombination site that can serve as a substrate for recombination with the first recombination site.

Recombinase polypeptides, and nucleic acids that encode the recombinase polypeptides, are described in the art and can be obtained using routine methods. These prokaryotic recombinases can be introduced into the eukaryotic cells that contain the recombination sites at which recombination is desired by any suitable method. For example, one can introduce the recombinase in polypeptide form, e.g., by microinjection or other methods. In presently preferred embodiments, however, a gene that encodes the recombinase is introduced into the cells. Expression of the gene results in production of the recombinase, which then catalyzes recombination among the corresponding recombination sites. One can introduce the recombinase gene into the cell before, after, or simultaneously with, the introduction of the exogenous polynucleotide of interest. In one embodiment, the recombinase gene is present within the vector that carries the polynucleotide that is to be inserted; the recombinase gene can even be included within the polynucleotide. In other embodiments, the recombinase gene is introduced into a transgenic eukaryotic organism, e.g., a transgenic plant, animal, fungus, or the like, which is then crossed with an organism that contains the corresponding recombination sites.

### Target Organisms

The methods of the invention are useful for obtaining integration and/or rearrangement of DNA in any type of eukaryotic cell. For example, the methods are useful for cells of animals, plants, and fungi. In some embodiments, the cells are part of a multicellular organism, e.g., a transgenic plant or animal. The methods of the invention are particularly useful in situations where transgenic materials are difficult to obtain, such as with transgenic wheat, corn, and animals. In these situations, finding the rare single copy insertion requires the prior attainment of a large number of independently derived transgenic clones, which itself requires great expenditure of effort.

Among the plant targets of particular interest are monocots, including, for example, rice, corn, wheat, rye, barley, bananas, palms, lilies, orchids, and sedges. Dicots are also suitable targets, including, for example, tobacco, cotton, apples, potatoes, beets, carrots, willows, elms, maples, roses, buttercups, petunias, phloxes, violets and sunflowers. Other targets include animal and fungal cells. These lists are merely illustrative and not limiting.

Constructs for Introduction of Exogenous DNA into Target Cells

The methods of the invention often involve the introduction of exogenous DNA into target cells. For example, nucleic acids that include one or more recombination sites are often introduced into the cells. The polynucleotide constructs that are to be introduced into the cells can include, in addition to the recombination site or sites, a gene or other functional sequence that will confer a desired phenotype on the cell.

In some embodiments, a polynucleotide construct that encodes a recombinase is introduced into the eukaryotic cells in addition to the recombination sites. The recombinase-encoding polypeptide can be included on the same nucleic acid as the recombination site or sites, or can be introduced into the cell as a separate nucleic acid. The present invention provides nucleic acids that include recombination sites, as well as nucleic acids in which a recombinase-encoding polynucleotide sequence is operably linked to a promoter which functions in the target eukaryotic cell.

Generally, a polynucleotide that is to be expressed (e.g., a recombinase-encoding polynucleotide or transgene of interest) will be present in an expression cassette, meaning that the polynucleotide is operably linked to expression control signals, e.g., promoters and terminators, that are functional in the host cell of interest. The genes that encode the recombinase and the selectable marker, will also be under the control of such signals that are functional in the host cell. Control of expression is most easily achieved by selection of a promoter. The transcription terminator is not generally as critical and a variety of known elements may be used so long as they are recognized by the cell.

A promoter can be derived from a gene that is under investigation, or can be a heterologous promoter that is obtained from a different gene, or from a different species. Where direct expression of a gene in all tissues of a transgenic plant or other organism is desired, one can use a “constitutive” promoter, which is generally active under most environmental conditions and states of development or cell differentiation. Suitable constitutive promoters for use in plants include, for example, the cauliflower mosaic virus (CaMV) 35S transcription initiation region and region VI promoters, the 1′- or 2′-promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis, Plant J., 11(5):1017-1027 (1997)), the mas, Mac or DoubleMac promoters (described in U.S. Pat. No. 5,106,739 and by Comai et al., Plant Mol. Biol. 15:373-381 (1990)) and other transcription initiation regions from various plant genes known to those of skill in the art. Such genes include for example, ACT11 from Arabidopsis (Huang et al., Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al., Plant Physiol. 104:1167-1176 (1994)), GPc1 from maize (GenBank No. X15596, Martinez et al., J. Mol. Biol 208:551-565 (1989)), and Gpc2 from maize (GenBank No. U45855, Manjunath et al., Plant Mol. Biol. 33:97-112 (1997)). Useful promoters for plants also include those obtained from Ti- or Ri-plasmids, from plant cells, plant viruses or other hosts where the promoters are found to be functional in plants. Bacterial promoters that function in plants, and thus are suitable for use in the methods of the invention include the octopine synthetase promoter, the nopaline synthase promoter, and the manopine synthetase promoter. Suitable endogenous plant promoters include the ribulose-1,6-biphosphate (RUBP) carboxylase small subunit (ssu) promoter, the (.alpha.-conglycinin promoter, the phaseolin promoter, the ADH promoter, and heat-shock promoters.

Promoters for use in E. coli include the T7, trp, or lambda promoters, a ribosome binding site and preferably a transcription termination signal. For eukaryotic cells, the control sequences typically include a promoter which option ally includes an enhancer derived from immunoglobulin genes, SV40, cytomegalovirus, etc., and a polyadenylation sequence, and may include splice donor and acceptor sequences. In yeast, convenient promoters include GAL1-10 (Johnson and Davies (1984) Mol. Cell. Biol. 4:1440-1448) ADH2 (Russell et al. (1983) J. Biol. Chem. 258:2674-2682), PHOS (EMBO J. (1982) 6:675-680), and MF.alpha. (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209).

Alternatively, one can use a promoter that directs expression of a gene of interest in a specific tissue or is otherwise under more precise environmental or developmental control. Such promoters are referred to here as “inducible” or “repressible” promoters. Examples of environmental conditions that may effect transcription by inducible promoters include pathogen attack, anaerobic conditions, ethylene or the presence of light. Promoters under developmental control include promoters that initiate transcription only in certain tissues, such as leaves, roots, fruit, seeds, or flowers. The operation of a promoter may also vary depending on its location in the genome. Thus, an inducible promoter may become fully or partially constitutive in certain locations. Inducible promoters are often used to control expression of the recombinase gene, thus allowing one to control the timing of the recombination reaction. Examples of tissue-specific plant promoters under developmental control include promoters that initiate transcription only in certain tissues, such as fruit, seeds, or flowers. The tissue-specific E8 promoter from tomato is particularly useful for directing gene expression so that a desired gene product is located in fruits. See, e.g., Lincoln et al. (1988) Proc. Nat'l. Acad. Sci. USA 84: 2793-2797; Deikman et al. (1988) EMBO J. 7: 3315-3320; Deikman et al. (1992) Plant Physiol. 100: 2013-2017. Other suitable promoters include those from genes encoding embryonic storage proteins. Examples of environmental conditions that may affect transcription by inducible promoters include anaerobic conditions, elevated temperature, or the presence of light. Additional organ-specific, tissue-specific and/or inducible foreign promoters are also known (see, e.g., references cited in Kuhlemeier et al (1987) Ann. Rev. Plant Physiol. 38:221), including those 1,5-ribulose bisphosphate carboxylase small subunit genes of Arabidopsis thaliana (the “ssu” promoter), which are light-inducible and active only in photosynthetic tissue, anther-specific promoters (EP 344029), and seed-specific promoters of, for example, Arabidopsis thaliana (Krebbers et al. (1988) Plant Physiol. 87:859). Exemplary green tissue-specific promoters include the maize phosphoenol pyruvate carboxylase (PEPC) promoter, small submit ribulose bis-carboxylase promoters (ssRUBISCO) and the chlorophyll a/b binding protein promoters. The promoter may also be a pith-specific promoter, such as the promoter isolated from a plant TrpA gene as described in International Publication No. WO93/07278.

Inducible promoters for other organisms include, for example, the arabinose promoter, the lacZ promoter, the metallothionein promoter, and the heat shock promoter, as well as many others that are known to those of skill in the art. An example of a repressible promoter useful in yeasts such as S. pombe is the Pnmt promoter, which is repressible by thiamine (vitamin B1).

Typically, constructs to be introduced into these cells are prepared using recombinant expression techniques. Recombinant expression techniques involve the construction of recombinant nucleic acids and the expression of genes in transfected cells. Molecular cloning techniques to achieve these ends are known in the art. A wide variety of cloning and in vitro amplification methods suitable for the construction of recombinant nucleic acids are well-known to persons of skill Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, used to transfect cells or incorporated into Agrobacterium tumefaciens to infect and transform plants. Where Agrobacterium is the means of transformation, shuttle vectors are constructed. Cloning in Streptomyces or Bacillus is also possible.

Selectable markers are often incorporated into the polynucleotide constructs and/or into the vectors that are used to introduce the constructs into the target cells. These markers permit the selection of colonies of cells containing the polynucleotide of interest. Often, the vector will have one selectable marker that is functional in, e.g., E. coli, or other cells in which the vector is replicated prior to being introduced into the target cell. Examples of selectable markers for E. coli include: genes specifying resistance to antibiotics, i.e., ampicillin, tetracycline, kanamycin, erythromycin, or genes conferring other types of selectable enzymatic activities such as beta-galactosidase, or the lactose operon. Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l. Acad. Sci. USA 78: 2072; Southern & Berg (1982) J. Mol. Appl. Genet. 1: 327).

Selection markers for plant cells often confer resistance to a biocide or an antibiotic, such as, for example, kanamycin, G 418, bleomycin, hygromycin, or chloramphenicol, or herbicide resistance, such as resistance to chlorsulfuron or Basta. Examples of suitable coding sequences for selectable markers are: the neo gene which codes for the enzyme neomycin phosphotransferase which confers resistance to the antibiotic kanamycin (Beck et al (1982) Gene 19:327); the hyg (hpt) gene, which codes for the enzyme hygromycin phosphotransferase and confers resistance to the antibiotic hygromycin (Gritz and Davies (1983) Gene 25:179); and the bar gene (EP 242236) that codes for phosphinothricin acetyl transferase which confers resistance to the herbicidal compounds phosphinothricin and bialaphos.

If more than one exogenous nucleic acid is to be introduced into a target eukaryotic cell, it is generally desirable to use a different selectable marker on each exogenous nucleic acid. This allows one to simultaneously select for cells that contain both of the desired exogenous nucleic acids.

Methods for Introducing Constructs into Target Cells

The polynucleotide constructs that include recombination sites and/or recombinase-encoding genes can be introduced into the target cells and/or organisms by any of the several means known to those of skill in the art. For instance, the DNA constructs can be introduced into plant cells, either in culture or in the organs of a plant by a variety of conventional techniques. For example, the DNA constructs can be introduced directly to plant cells using biolistic methods, such as DNA particle bombardment, or the DNA construct can be introduced using techniques such as electroporation and microinjection of plant cell protoplasts. Particle-mediated transformation techniques (also known as “biolistics”) are described in Klein et al., Nature, 327:70-73 (1987); Vasil, V. et al., Bio/Technol. 11:1553-1558 (1993); and Becker, D. et al., Plant J., 5:299-307 (1994). These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants, bacteria, fungi, algae, intact animal tissues, tissue culture cells, and animal embryos. One can employ electronic pulse delivery, which is essentially a mild electroporation format for live tissues in animals and patients. Zhao, Advanced Drug Delivery Reviews 17:257-262 (1995).

Other transformation methods are also known to those of skill in the art. Microinjection techniques are known in the art and well described in the scientific and patent literature. The introduction of DNA constructs using polyethylene glycol (PEG) precipitation is described in Paszkowski et al., EMBO J. 3:2717 (1984). Electroporation techniques are described in Fromm et al., Proc. Natl. Acad. Sci. USA, 82:5824 (1985). PEG-mediated transformation and electroporation of plant protoplasts are also discussed in Lazzeri, P., Methods Mol. Biol. 49:95-106 (1995). Methods are known for introduction and expression of heterologous genes in both monocot and dicot plants. See, e.g., U.S. Pat. Nos. 5,633,446, 5,317,096, 5,689,052, 5,159,135, and 5,679,558; Weising et al. (1988) Ann. Rev. Genet. 22:421-477. Transformation of monocots in particular can use various techniques including electroporation (e.g., Shimamoto et al., Nature (1992), 338:274-276); biolistics (e.g., European Patent Application 270,356); and Agrobacterium (e.g., Bytebier et al., Proc. Nat'l Acad. Sci. USA (1987) 84:5345-5349).

For transformation of plants, DNA constructs may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-meditated transformation techniques are well described in the scientific literature. See, for example, Horsch et al. Science, 233:496-498 (1984), Fraley et al., Proc. Natl. Acad. Sci. USA, 80:4803 (1983), and Hooykaas, Plant Mol. Biol., 13:327-336 (1989), Bechtold et al., Comptes Rendus De L Academie Des Sciences Serie Iii-Sciences De La Vie-Life Sciences, 316:1194-1199 (1993), Valvekens et al., Proc. Natl. Acad. Sci. USA, 85:5536-5540 (1988). For a review of gene transfer methods for plant and cell cultures, see, Fisk et al., Scientia Horticulturae 55:5-36 (1993) and Potrykus, CIBA Found. Symp. 154:198 (1990).

Other methods for delivery of polynucleotide sequences into cells include, for example liposome-based gene delivery (Debs and Zhu (1993) WO 93/24640; Mannino and Gould-Fogerite (1988) BioTechniques 6(7): 682-691; Rose U.S. Pat No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; All et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

Methods by which one can analyze the integration pattern of the introduced exogenous DNA are well known to those of skill in the art. For example, one can extract DNA from the transformed cells, digest the DNA with one or more restriction enzymes, and hybridize to a labeled fragment of the polynucleotide construct. The inserted sequence can also be identified using the polymerase chain reaction (PCR). See, e.g., Sambrook et al., Molecular Cloning—A Laboratory Manual, Cold Spring Harbor Laboratory, Cold Spring Harbor, N.Y., 1989 for descriptions of these and other suitable methods.

### Regeneration of Transgenic Plants and Animals

The methods of the invention are particularly useful for obtaining transgenic and chimeric multicellular organisms that have a stably integrated exogenous polynucleotide or other stable rearrangement of cellular nucleic acids. Methods for obtaining transgenic and chimeric organisms, both plants and animals, are well known to those of skill in the art.

Transformed plant cells, derived by any of the above transformation techniques, can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, Macmillian Publishing Company, New York (1983); and in Binding, Regeneration of Plants, Plant Protoplasts, pp. 21-73, CRC Press, Boca Raton, (1985). Regeneration can also be obtained from plant callus, explants, somatic embryos (Dandekar et al., J. Tissue Cult. Meth., 12:145 (1989); McGranahan et al., Plant Cell Rep., 8:512 (1990)), organs, or parts thereof. Such regeneration techniques are described generally in Klee et al., Ann. Rev. of Plant Phys., 38:467-486 (1987).

The methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994. Transgenic fish having specific genetic modifications can also be made using the claimed methods. See, e.g., Iyengar et al. (1996) Transgenic Res. 5: 147-166 for general methods of making transgenic fish.

One method of obtaining a transgenic or chimeric animal having specific modifications in its genome is to contact fertilized oocytes with a vector that includes the polynucleotide of interest flanked by recombination sites. For some animals, such as mice fertilization is performed in vivo and fertilized ova are surgically removed. In other animals, particularly bovines, it is preferably to remove ova from live or slaughterhouse animals and fertilise the ova in vitro. See DeBoer et al., WO 91/08216. In vitro fertilization permits the modifications to be introduced into substantially synchronous cells. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. If desired, the presence of a desired exogenous polynucleotide in the embryo cells can be detected by methods known to those of skill in the art. Methods for culturing fertili7ed oocytes to the pre-implantation stage are described by Gordon et al. (1984) Methods Enzymol. 101: 414; Hogan et al. Manipulation of the Mouse Embryo: A Laboratory Manual, C.S.H.L. N.Y. (1986) (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim. Sci. 66: 947-953 (ovine embryos) and Eyestone et al. (1989) J. Reprod. Fert. 85: 715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Alternatively, the methods can be used to obtain embryonic stem cells (ES) that have a single copy of the desired exogenous polynucleotide. These cells are obtained from preimplantation embryos cultured in vitro. See, e.g., Hooper, M L, Embryonal Stem Cells: Introducing Planned Changes into the Animal Germline (Modern Genetics, v. 1), Int'l. Pub. Distrib., Inc., 1993; Bradley et al. (1984) Nature 309, 255-258. Transformed ES cells are combined with blastocysts from a non-human animal. The ES cells colonize the embryo and in some embryos form the germ line of the resulting chimeric animal. See Jaenisch, Science, 240: 1468-1474 (1988). Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

### EXAMPLES, EXPERIMENTS, AND ASSAYS

The following examples and experiments are intended to be illustrative only, and not intended to set forth the scope of the claimed invention or to limit it in any way.

### Materials and Methods

Biological strains. Schizosaccharomyces pombe strain FY527 (h⁻ ada6-M216 his3-D1 leu-32 ura-D18) was obtained from S. Forsburg (Salk Institute, San Diego). Strain Sp223 (h⁻ leu1.32 ura4.294 ade.216) have been previously described (Ortiz et al., 1992). Arabidopsis Columbia ecotype and tobacco (Nicotiana tabacum Wi38) are laboratory stocks. Human cell line HEK293 is described previously (Simmons N L., 1990). Recombinase genes of the CinH (pKLH205.63), parA (pWIS17), Tn1721 (pRU576.8), Tn5053 (pKLH53.6) Tn21, Tn402, and Tn501 systems were obtained from Gennady Kholodii (Russian Academy of Sciences; Moscow, Russia), U153 system (genomic DNA of Listeria monocytogenes phage phiCU-51153/95, respectively) from Richard Calender (University of California; Berkeley, USA), Bxb1 system (pMA1) from Graham Hatfull (University of Pittsburgh; Pittsburgh, USA), and TP901 system (pBC170) from Karen Hammer (Technical University of Denmark; Lyngby, Denmark.

Recombinase genes and recombination sites. Recombinase ORFs, and the recombination sites for CinH, ParA, Tn1721, Tn5053, and U153 (attB) were amplified (Turbo Pfu, Stratagene) through PCR (primers listed below). PCR primers in powder form (Operon) were resuspended in 1 ml of TE and incubated at 42° C. for 30 minutes. OD260/280 was used to determine concentration and purity. Concentrations were adjusted to 50 ng/ml and aliquots stored at −80° C. until use. Recombination sites for Bxb1, PhiC31, TP901-1, and U153 (attP) were assembled with DNA oligonucleotides. Synthetic DNA were resuspended in 1 ml of TE and incubated at 42° C. for 30 minutes. The annealed oligos were treated with a kinase enzyme to phosphorylate the 5′ ends. Complementary oligos were combined in equal molar ratios, incubated at 90° C. for 10 minutes, and cooled slowly to anneal.

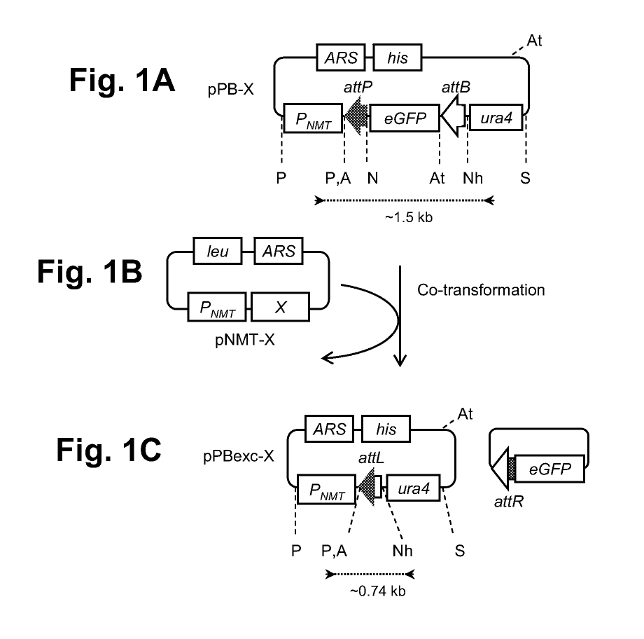

The generic recombinase expression construct is pNMT-X (FIG. 1B), where X represents the recombinase gene under the control of the thiamine repressible promoter P_{NMT}. The recombinase genes were amplified by Turbo Pfu (Stratagene), and inserted into plasmid pNMT1-TOPO (Invitrogen). PCR amplifications conditions: for small resolves: 94° C., 5min; 94° C., 30 s; 54° C., 30 s; 72° C., 45 s (20 cycles); 72° C., 5 min with a 22° C. hold; for large resolvases: 94° C., 5 min; 94° C., 30 s; 54° C., 30 s; 72° C., 2 min (20 cycles); 72° C. 5 min (1) with a 22° C. hold. ORF fragments were sequenced for confirmation.

Excision detection system constructs. The generic excision detection construct, pPB-X (FIG. 1A), where X represents the recombination sites used, contains his3 and ARS for selection and replication, respectively, in S. pombe. These constructs were derived from pLT43 (Thomason et.al., 2001). First, the NotI site of pLT43 was removed by cleavage with NotI, the staggered ends filled by DNA polymerase I (Klenow fragment), and blunt end closed with DNA ligase. The SacI flanked phiC31 ORF was then replaced with a first lox site flanked by AscI and NotI. The insertion also leaves the downstream SacI site intact. The construct was then treated with NotI and SacI to permit insert of a second lox site, which brings along flanking BclI and NheI sites. A NheI to SacI ura4 fragment was then inserted into the NheI and SacI sites to place ura4 downstream of the second lox site, and a NotI to EagI eGFP (enhanced green florescent protein) coding region was inserted into the NotI site to place eGFP between the first and the second lox sites to yield pPB-Cre. The NheI to SacI ura4 fragment was derived from prior modification by PCR to include an NheI site 5′ to the ATG and a SacI site 3′ of the polyA tail, and cloned into the pNMT-TOPO vector. Likewise, the NotI to EagI eGFP fragment was derived from prior modification by PCR to include a NotI site 5′ to the ATG and an EagI site 3′ of the polyA tail, and cloned into pNMT-TOPO. Other pPB-X constructs were derived from pPB-Cre by replacement of first and second lox sites with system unique recombination sites flanked by AscI and NotI or BclI and NheI. Replacement of the ura4 proximal lox site with a new recombination site through cleavage by BclI and NheI also shortened the eGFP fragment by removal of a 277 by transcription terminator region, which was not necessary for eGFP expression in S. pombe.

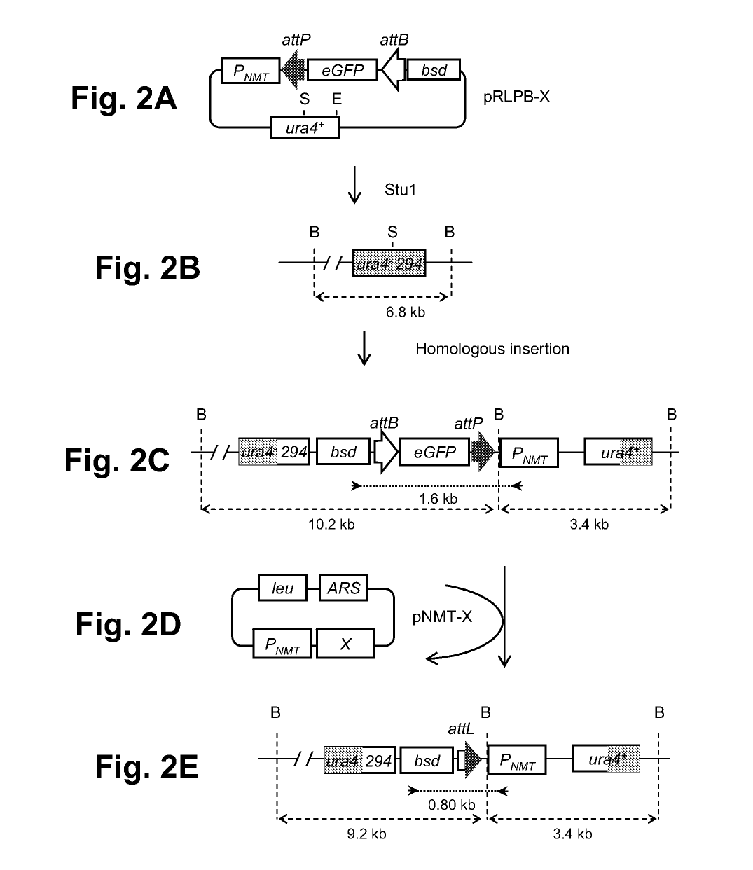

The generic excision detection construct for testing recombination of S. pombe nuclear DNA is pRLPB-X (FIG. 2A), where X represents the recombination sites used. These pRLPB-X constructs were derived from pRIP4X (S. Forsburg) and contain a 1.8 kb ura4 gene fragment for homologous insertion into the corresponding ura4.294 allele in the genome (FIG. 2B). The plasmid pRIP4X was cleaved with endonuclease EcoRI to remove the ARS and the linearized backbone was recircularized in the presence of an EcoRI/AscI/BglII/NheI/SacI/EcoRI linker to yield pRL with the linker in the orientation where AscI is promixal to P_{NMT} promoter. A bsd (blasticidine resistance gene) ORF was amplified by PCR from pEF/Bsd (Invtrogen) to contain 5′ NheI and 3′ SacI staggered ends, and inserted into pRL cleaved with NheI and SacI sites. The resulting construct, pRLB, was cleaved with AscI and NheI to allow insertion of an AscI/PstI/attP/eGFP/attB/NheI fragment from the pPB-X constructs. Each pRLPB-X construct was linearized at the Stul site of the 1.8 kb ura4 gene, transformed into Sp223 for uracil prototrophic colonies. Homologous insertions were screened by Southern blotting and used for testing site-specific recombination of DNA in the nuclear genome.

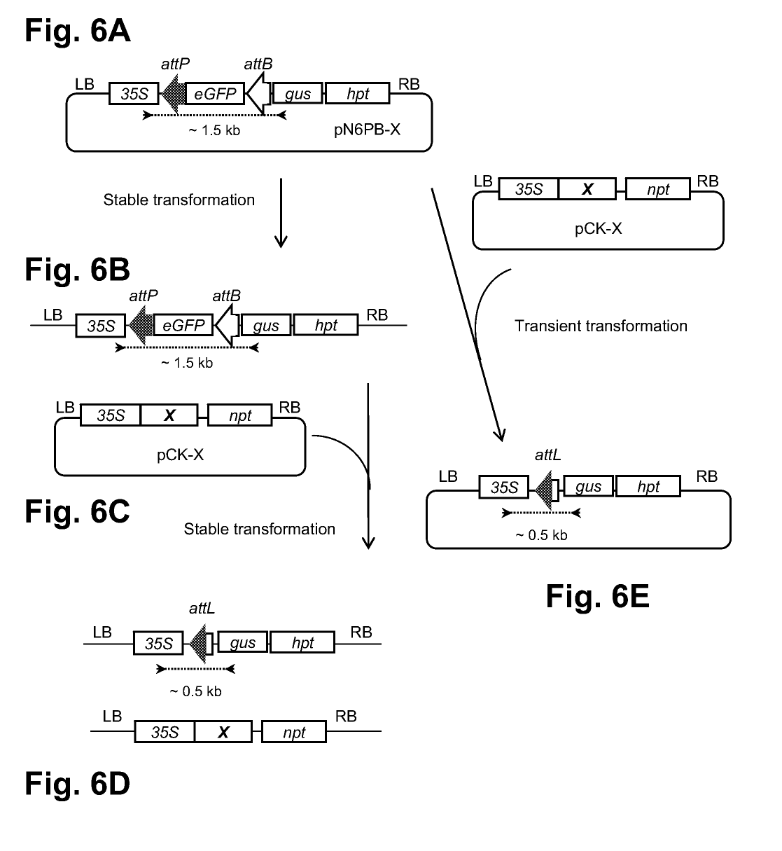

The generic constructs for site-specific excision in plant cells are pN6PB-X and pCK-X (FIG. 6A, C), where “X” designates the recombination system. pN6PB-X was derived from pCambia1301 (Cambia, Canberra) which contains a hygromycin resistance gene for plant transformation. Construct pCambia1301 was cut with NcoI between 35S and gus and filled with a AscI/Spel linker. The derivative was then cut with AscI and Spel to insert in an AscI/attP/eGFP/attB/NheI fragment from the pPB-X constructs. The pCK-X vectors were derived from pCambia2300 (Cambia, Canberra) and pKar6 (R. Blanvillian, PGEC). Construct pKar6 was cut with NcoI, and reclosed in the presence of an AscI linker. The derivative was cut with AscI and XbaI and ligated to an AscI/ORF/SpeI fragment from pNMT-X to yield pK-X. This places the recombinase coding region downstream of d35S (a CaMV 35S RNA promoter, double enhancer element version) and upstream of a 35S terminator (35S-3′) The d35S-X-35S-3′ fragment from pK-X was then retrieved as a HindIII fragment and inserted into pCambia2300, which contains a kanamycin resistance gene for plant transformation.

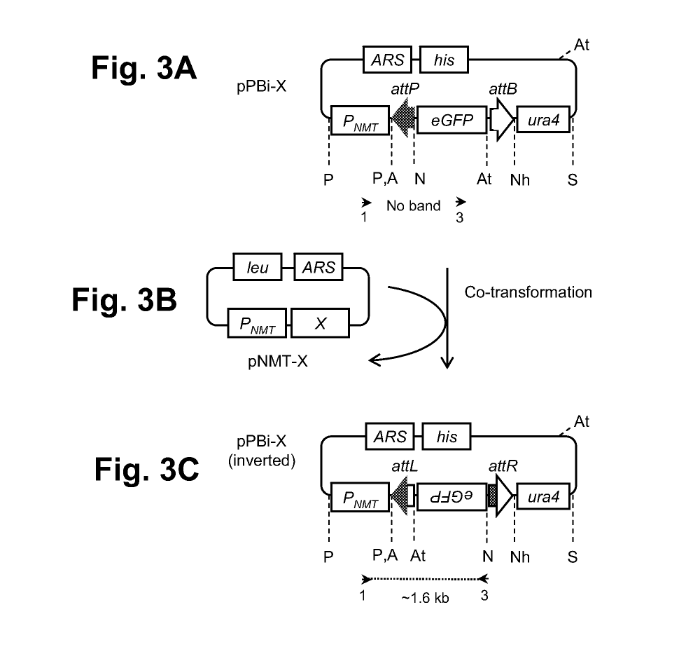

Inversion detection system constructs. The ura4 proximal recombination site in each of the pPB-X plasmids was inverted by replacing the recombination site with the same recombination site that is situated in the opposite orientation with respect to the flanking BclI and NheI sites. This yielded the generic test construct pPBi-X, where X represents the recombination system (FIG. 3A). Cleavage by BclI also removed the eGFP polyA tail (277 bp) from the eGFP fragment, which was not needed for expression in S. pombe. Therefore the polyA region was not reintroduced into the construct. Recombination sites longer than 100 by were produced by PCR, while the smaller recombination sites were synthetic DNA. Amplification of recombination sites was carried out using Turbo Pfu (Stratagene).

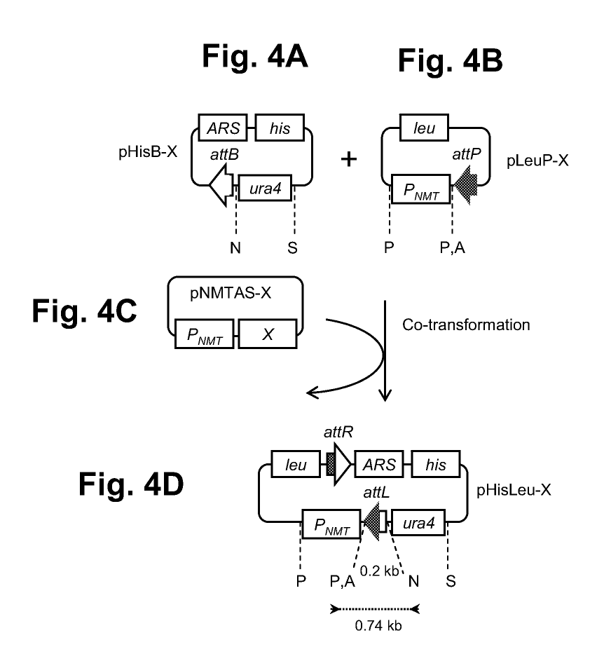

Co-integration detection system. The receptor pHisB-X constructs (FIG. 4B) were derived from the corresponding pPB-X constructs by removal of the P_{NMT}-first recombination site-eGFP fragment. pPB-X was cleaved with PstI and BclI, followed by treatment with DNA polymerase I (Klenow fragment), and then with DNA ligase. The donor pLeuP-X constructs (FIG. 4A) were generated by inserting the first recombination site (complementary or identical to the one in pHisB-X, such as attP or res, respectively) into a pNMT-TOPO derivative that had been modified to contained unique sites AscI and NotI 3′ to the promoter P_{NMT}. This derivative was further modified by treatment with SalI and AfeI, DNA polymerase I (Klenow fragment), and DNA ligase to remove the ARS. The recombinase expression constructs, pNMTAS-X (FIG. 4C), were generated by removal of the leu and ARS sequences from pNMT-X constructs, through treatment of pNMT-X with Afel and Stul, followed by DNA ligase.

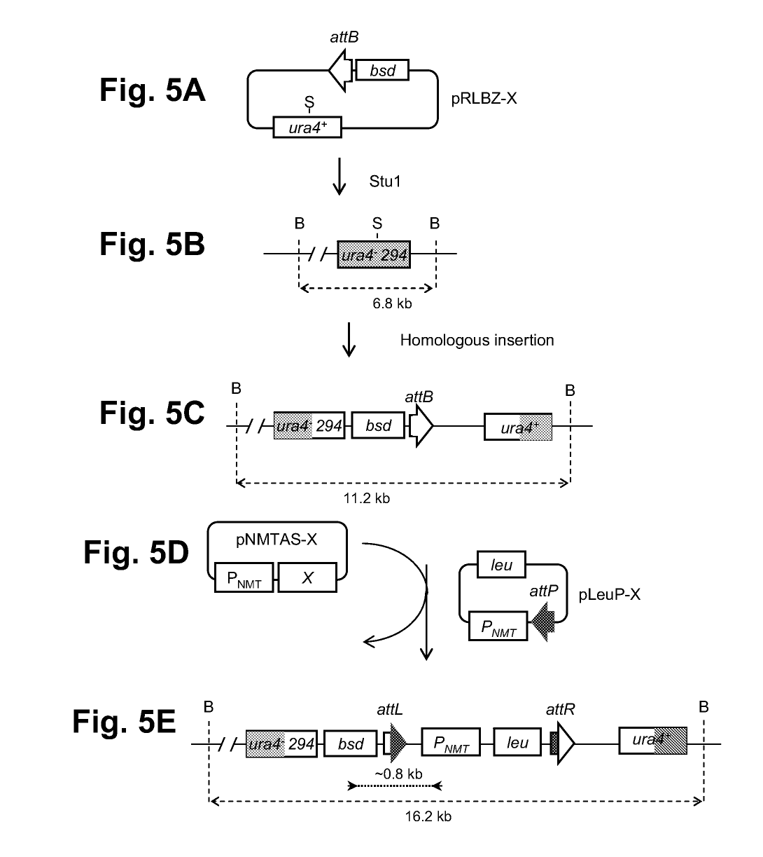

The pRLBZ-X constructs (FIG. 5A) for homologous insertion into the S. pombe genome were derived from pRLB. The pRLB plasmid was cleaved with BglII and NheI and ligated to a BclI/AatII/attB/NheI linker, where attB is from the corresponding “X” recombination system. The resulting intermediate plasmid was then cleaved with AscI and AflII to remove the PNMTFi primer-binding site in P_{NMT} upstream of the attB site, the staggered ends were made blunt by Klenow treatment, and the linear DNA recircularized to form pRLBZ-X. Each pRLBZ-X construct was linearized at the Stul site within the 1.8 kb ura4 gene fragment and transformed into Sp223 for uracil prototrophic colonies. Homologous insertions were screened by Southern blotting and used for testing site-specific insertion of DNA into the recombination site (attB) site in the nuclear genome.

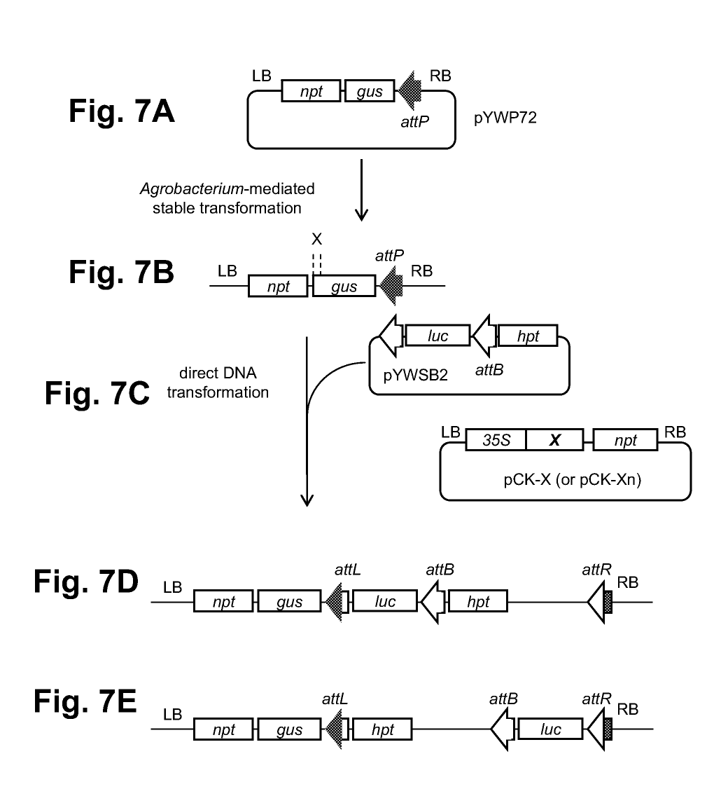

The constructs for testing in animal cells were derived from pEGFP-N and pQCEIN (Clontech). The Clontech vector pQCEIN was cleaved with SpeI and Agel to remove the retroviral 5′LTR and Psi sites. The remaining vector fragment was ligated with an “X” attB site containing SpeI and AgeI sites. The final vector pBEIN-X contained a “X” attB site followed downstream by a promoterless eGFP ORF, followed by an IRES-Neo gene for selection (FIG. 7A). The Clontech vector pEGFP-N was cleaved with AgeI and NotI to remove the eGFP ORF and replace it with an “X” attP site with AgeI and NotI staggered ends. This places the attP site downstream of a CMV promoter/enhancer to form pQCAP-X, where X indicates the recombination system (FIG. 7B). The “X” recombinase plasmid was produced by inserting a PCR-amplified DNA fragment containing the “X” recombinase ORF and inserting it directly into Novagen's LIC/EK kit to yield pLIC-X, where X indicates the recombination system (FIG. 7C). The recombinase ORF is flanked by a CMV promoter/enhancer and a beta globin polyA tail.

S. pombe transformation. DNA constructs were introduced into S. pombe FY527 or Sp223 by Li-acetate treatment or electroporation (Gietz and Woods, 2002). For electroporation, a 4 ml overnight culture OD₆₀₀˜0.7 was washed 3× with 1.2 M sorbitol and resuspended in 40 μl of 1.2 M sorbitol. The cells were combined with DNA in a Biorad 0.1 cm cuvette and electroporated (BioRad Gene pulser system) at 1.6 kV 200 ohm and 25 uF for ˜5 ms. Cells were immediately transferred to 160 ul of 1.2 M sorbitol and incubated on ice for 10 min. 200 μl of cells were spread onto EMM (Bio 101) plates supplemented with 2 mM adenine and uracil and incubated at 30° C. for 7 days.

Transfection of plant protoplasts. Protoplasts were transformed using a modified electroporation procedure (Dale and Ow, 1990; Morgan and Ow, 1995). Each cuvette (Biorad No. 165-2085) contained ˜1×10⁶/ml of cells, ˜80 ug of sheared salmon sperm, and various amounts (from 0 to 10 ug) of the transfecting DNA. Cells were then incubated at 26° C. for two to three days in the dark prior to DNA extraction.

Transfection of mammalian cells. Human HEK293 cells were seeded into a 6-well plate at 1×10⁶ per well in DMEM, 10% FBS and 1% pen/strep, incubated overnight at 37° C. and 5% CO₂. Prior to cell transfection, 3 ug of DNA was pre-incubated with 9 ul of FuGENE6 in 100 ul DMEM at room temperature for 20 minutes. DNA was then added drop-wise to respective HEK293 containing wells. Cells were incubated for 48 hours prior to UV-microscopy and FACS analysis.

Plant transformation. Arabidopsis and tobacco were transformed by Agrobacterium GV3101 by standard procedures such as described previously (Dale and Ow, 1991; Fisher and Guiltiman, 1995; Valvekens et al., 1988; Day et al., 2000).

Molecular analysis of site-specific recombination. Individual yeast colonies were swiped with a toothpick (˜0.3 μl volume of DNA) and resuspended in ˜25 μl of the following solution: 2.5 μl Taq buffer 10×, 2.0 μl MgCl₂, 1.0 μl dNTPs (2 mM), 0.5 μl DMSO, 0.5 μl 1% Triton X, 0.2 μl Taq (Promega), 2 μl of applicable primers, and 16 μl MQ H₂O. Ericomp PCR thermocycler (San Diego, Calif.) conditions: 95° C. 5 min; 94° C. 30 s, 60.5° C. 30 s (or 58° C. 30 s for inversion reactions), 72° C. 1.5 min (35 cycles); 72° C. 5 min with a 22° C. hold. A 0.8% agarose gel was used to visualize results. For excision or integration, recombination shows a 0.74 kb fragment. PCR primers used (1.0 μl of each at 50 ng/ml): Forward primer PNMTFi, specific for the P_{NMT} promoter, and reverse primer UraUP, specific for the ura4 ORF. For inversion, recombination shows a 1.6 kb fragment with primers PNMTFi and EGFP F (primers 1 and 3 in FIG. 5, respectively). Plasmid DNA retrieved from S. pombe for transformation into E. coli were prepared as described in “Rapid Isolation of Plasmid DNA from Yeast” Short Protocols In Molecular Biology 4^{th} edition). The analysis of recombination in plant cells was similar conducted by PCR. DNA was extracted from the protoplasts using the Gentra Genomic DNA extraction Kit. The DNA was resuspended in 50 ul of H₂O and a 3 ul aliquot was used for PCR amplification by the primers indicated in the Figures.

Southern blot analysis was conducted by standard procedures. Genomic DNA was cleaved with EcoRI, EcoRV or BamHI. DNA was fractionated by gel electrophoresis, transferred to nylon membrane, hybridized with radioactive ura4 DNA probes, and exposed to x-ray film.

### Recombination Systems Capable of Inducing an Intra-Molecular Recombination Event.

DNA Excision Assay. To determine whether a prokaryotic recombination system has activity in a eukaryotic cell, a first site-specific recombination assay was conducted as depicted in FIG. 1. A Leu⁺ plasmid, pNMT-X, where X represents the particular recombinase, places the recombinase coding-region under the control of a thiamine-repressible P_{NMT} promoter. Depending on the particular recombination system, the recombinase may be known also as a resolvase or an integrase. A His⁺ plasmid, pPB-X, where X represents the particular recombination system, contains a unique set of recombination sites flanking an eGFP (enhanced green florescence protein) fragment consisting of the coding region and transcription terminator. This eGFP fragment is situated between P_{NMT} and an ura4 coding region. Depending on the particular recombination system, the recombination sites may also be known by their specific names, such as attachment (att) or resolution (res) sites. Both plasmids were introduced into S. pombe FY527 (h⁻ ada6-M216 his3-D1 leu-32 ura-D18) selecting for Leu⁺ His⁺ colonies. In the case of successful site-specific recombination, recombinase produced from pNMT-X recombines the set of recombination sites in pPB-X. Excision of the eGFP fragment fuses P_{NMT} with ura4 (FIG. 1C). Hence, the experimental design was intended to provide a phenotypic assay for site-specific recombination, the loss of eGFP expression concomitant with the gain of Ura⁺ autotrophy. However, this expectation assumes that the hybrid recombination site formed by the recombination reaction will not interfere with expression of the downstream ura4 gene. Whether that be the case will depend on the sequence and length of the given hybrid recombination site, as well as the translational tolerance of the cell type for the intervening DNA (recombination site sequence).

In light of this uncertainty with the phenotype assay, site-specific recombination was also assayed biochemically by a PCR analysis on individual Leu⁺ His⁺ colonies. As illustrated in FIG. 1, a primer pair amplifies a product of ˜1.5 kb in pPB-X, but a smaller product of ˜0.74 kb from the excision derivative of pPB-X (the exact sizes of these PCR products differ slightly depending on the length of the particular recombination site used in pPB-X). Representative excision products (pPBexc-X, FIG. 1C) are then retrieved from S. pombe, transformed into E. coli, where the DNA was structurally analyzed by endonuclease digestion.

Recombination Systems where the Recombination Sites are Identical: Cre-lox, CinH, ParA, Tn1721, Tn5053.

Cre-lox. The Cre recombinase from bacteriophage P1, a member of the tyrosine subfamily of recombinases, catalyzes reversible recombination between 34 bp recombination sites known as lox (locus of crossover). The Cre-lox system is known to recombine in eukaryotic cells. Using it as a positive control, the PCR assay showed a distinct shift in band size from 1.4 kb to 0.74 kb, consistent with Cre mediated excision of the eGFP open reading frame (ORF) from pPB-Cre. This event was accompanied by an Ura⁺ phenotype in the Leu⁺ His⁺ colonies. Determining the PCR product corresponding to the excision event confirmed the precise recombination between the intramolecular lox sites. Based on the PCR of 10 colonies in each of 3 independent experiments, excision was detected in 29 out of 30 Leu⁺ His⁺ colonies. Moreover, the 0.74 kb excision product was the only band detected. This indicates that the reaction reached completion 7 days after plating (See Table 1). The single colony lacking site-specific recombination was not analyzed further, as it may represent other events such as mutations in the recombinase gene or the recombination sites.

TABLE 1
Intramolecular excision in S. pombe

| Recombi- | | | | Leu⁺ His⁺ | cfu with | Estimated |
|---|---|---|---|---|---|---|
| nation | | | His⁺ Leu⁺ | cfu/total cfu | excision/cfu | completion |
| system | Transfected constructs | | cfu (10²) ^{a} | (10⁻⁴) ^{a} | analyzed ^{b} | rate ^{c} |
| Cre | pPB-Cre | pNMT-Cre | 14.5 ± 0.81 | 5.18 ± 0.56 | 29/30 | 100% |
| CinH | pPB-CinH | pNMT-CinH | 12.2 ± 2.8 | 6.01 ± 3.29 | 29/30 | 95% |
| parA | pPB-ParA | pNMT-ParA | 6.7 ± 2.5 | 4.93 ± 0.66 | 29/30 | 85% |
| Tn1721 | pPB-Tn1721 | pNMT-Tn1721 | 5.8 ± 4.7 | 4.26 ± 2.77 | 29/30 | 70% |
| Tn5053 | pPB-Tn5053 | pNMT-Tn5053 | 17.6 ± 8.7 | 11.4 ± 10.0 | 27/30 | 50% |
| phiC31 | pPB-phiC31 | pNMT-phiC31 | 9.3 ± 2.8 | 3.28 ± 0.86 | 29/30 | 80% |
| TP901-1 | pPB-TP901 | pNMT-TP901 | 12.0 ± 2.0 | 4.25 ± 0.59 | 29/30 | 80% |
| Bxb1 | pPB-Bxb1 | pNMT-Bxb1 | 15.1 ± 1.4 | 5.37 ± 0.58 | 29/30 | 95% |
| U153 | pPB-U153 | pNMT-U153 | 24.3 ± 5.4 | 8.81 ± 3.1 | 28/30 | 35% |

^{a} cfu = colony forming units; mean ± SD from 3 independent experiments.
^{b} Detection of excision by PCR. Data from 10 colonies analyzed for each of 3 independent experiments.
^{e.} Estimate based on PCR band intensities.

CinH. The CinH recombination system is from Acinetobacter sp. ED45-25 plasmids pKLH2, pKLH204 and pKLH205 (Kholodii, 2001). The recombinase of this system belongs to the class of small serine resolvases that include the prototypical Tn3 (Watson et al., 1996). In its plasmid genome, the 580 by CinH recombinase gene is located between two directly repeated recombination sites known as RS2. The two identical RS2 sites are each 113 by in length. Like the Cre-lox system, the product sites derived from recombination are identical to the substrate sites. Unlike the Cre-lox system, however, this reaction is not reversible. Like most recombinases of the small serine recombinase subfamily, CinH recombinase is incapable of mediating inversion or integration. This may be due to the selectivity of synapsis or “topological filter” seen among these enzymes (Watson et al., 1996). It has been suggested that recombination site pairwise relationships are topologically restricted, and will allow only the directly repeated sites on the same DNA strand to generate the conformation required to produce a productive synaptoneal complex.

The pPB-CinH construct failed to express eGFP. This was not due to a defective eGFP fragment, as this same fragment when transferred to another vector was functional. Cells co-transformed with pPB-CinH and the pNMT-CinH also failed to produce Ura⁺ colonies. Hence, it is likely that the CinH recombination site interferes with downstream gene expression. Indeed, the PCR assay on Leu⁺ His⁺ colonies showed a distinct shift from a 1.5 kb product before recombination to an expected 0.74 kb product after recombination, similar to that for the Cre-lox system. This is consistent with deletion of the intervening eGFP ORF. Based on the PCR analysis of 10 colonies in each of 3 independent experiments, excision was detected in 99% of the Leu⁺ His⁺ colonies assayed. However, the CinH excision was only about ˜95% complete by day 7, as judged by the relative intensities of the excised (˜0.74 kb) to the unexcised (˜1.5 kb) bands (Table 1). Representative excision derivatives of the detection construct rescued into E. coli confirmed site-specific deletion of eGFP, as cleavage with AscI and SacI released a 1.8 kb band from the parental construct pPB-CinH, but a new 0.96 kb band from pPBexe-CinH was also identified. Precise site-specific recombination was confirmed from sequencing both the 0.74 kb PCR product DNA and the product recombination site derived from pPBexe-CinH.

ParA. The ParA system is from a plasmid operon parCBA, which along with parDE, is responsible for the maintenance of broad host range plasmids RK2 and RP4. Operon parCBA resolves plasmid multimers to monomers to insure that each daughter cell can have a copy of the plasmid, while parDE kills the cells that do not contain a plasmid copy (postsegregational killing). The 679 by parA encoded recombinase recognizes a pair of identical 133 by recombination sites termed MRS (multimer resolution site), which is located between the parCBA and parDE operons (Roberts et al., 1994; Gerlitz et al., 1990; Sobecky et al., 1996). The ParA system shares similar properties with the CinH system. The ParA recombinase is also a member of the small serine resolvase subfamily, and while it can recombine MRS sites to cause DNA deletion, it cannot cause inversion or co-integration reactions.

Cotransformation by the two-plasmid excision detection system described above did not confer an Ura⁺ phenotype in the Leu⁺ His⁺ colonies. However, the PCR assay detected a shift in band size from ˜1.5 kb to ˜0.74 kb, which was seen in 99% of the colonies analyzed. This is consistent with deletion of the intervening eGFP ORF. The lack of an Ura⁺ phenotype in the Leu⁺ His⁺ colonies is probably due to interference of downstream gene expression by the 133 by MRS site. The PCR assay also detected bands representing unexcised molecules. Based on the relative intensities of the excised and unexcised bands, excision was estimated to be ˜85% by day 7 after plating (See Table 1). Representative recombinant plasmids rescued into E. coli showed a drop in band size from 1.8 kb to 0.96 kb upon cleavage with AscI and SacI, and from 1.0 kb to 0.2 kb when cleaved with AscI and NheI. This corresponds to a deletion of the eGFP ORF. The DNA sequence of the ˜0.74 kb PCR product, as well as the product recombination site derived from pPBexe-ParA, showed that the excision event occurred in a conservative manner.

Tn1721. Tn1721 was identified as a constituent of the conjugative plasmid pRSD1 (Burkardt et al., 1978). Isolated as part of a 10.7 kb tetracycline resistant transposable element in E. coli the Tn1721 transposon is capable of forming a multiple duplication of a 5.3 kb region that encompasses the tetracycline resistance gene (Schmitt et al. 1979). Tn1721 alone is a 3.8 kb transposon that encodes a transposase (tnpA), a resolvase (tnpR), and a recombination site (res) (Ferreira et al., 2002). Translocation of Tn1721 generates a five base pair direct repeat (TCCTT-res site-TCCTT) at the respective site of insertion. The tnpR resolvase, encoded by a 570 by ORF, is a member of the small serine recombinase subfamily that includes the prototypical Tn3 (Watson et al., 1996) and recognizes a pair of 120 by recombination (res) sites. In vitro data suggests that only directly repeated res sites are capable of recombination. Supercoiled DNA was also a requirement for recombination although the recombinase appears capable of binding the res sites of linear DNA (Rogowsky and Schmitt, 1985). When sites were oriented as an inverted repeat, inversion was not detected, consistent with similar findings with Tn3 and gamma delta resolvases (Reed, 1981; Krasnow and Cozzarelli, 1983). In vivo data in bacteria, however, differed slightly from the in vitro data and suggests that inversion can occur at low efficiency (Altenbuchner and Schmitt, 1983).

Cotransformation by the two-plasmid excision detection system described above failed to yield an Ura⁺ phenotype in the Leu⁺ His⁺ hcolonies. Lack of an Ura⁺ phenotype in the Leu⁺ His⁺ colonies is probably due to interference of downstream gene expression by the 120 bp res site. A shift in band size from ˜1.5 kb to ˜0.74 kb was detected in 99% of the colonies examined by PCR, which is consistent with deletion of the intervening eGFP ORF. Based on relative intensities of the two PCR products, the excision was found to be ˜70% complete by day 7 (See Table 1). Representative recombinant plasmids rescued into E. coli produced the expected restriction pattern, in that cleavage by AscI and SacI revealed a new 0.96 kb fragment, and AscI and NheI showed a 0.2 kb band. The sequence of the ˜0.74 kb PCR product as well as the product recombination site derived from pPbexc-Tn1721 showed that the excision event occurred in a conservative manner.

Tn5053. The transposon, Tn5053, was isolated from a mercury-resistant Xanthomonas sp. W17 strain originating from a Khaidarkan, Kirghizia mercury mine. The 8.4 kb transposon is bracketed by 25 base-pair inverted repeats that have no sequence homology to known mercury resistance transposons (Kholodii et al., 1993). Tn5053 comprises of a mercury-resistance module and a transposition module. The mercury-resistance module carries a merRTPFAD operon, and appears to be a single-ended relic of a transposon closely related to the classical mercury-resistance transposons Tn21 and Tn501. The transposition module of Tn5053 is bounded by 25 by terminal inverted repeats and contains four genes involved in transposition, tniA, tniB, tniQ, and tniR. Transposition of Tn5053 occurs via cointegrate formation mediated by the products of tniA, tniB and tniQ, followed by cointegrate resolution at the ˜200 by res site located upstream of tniR. This resolution event is catalyzed by the Tn5053 recombinase, a small-serine recombinase encoded by the 615 bp tniR gene (Kholodii et al., 1995). When tested in bacteria, excision and inversion, although inversion occurs at a reduced rate, are possible with properly oriented res sites (Kholodii, 1995).

Cotransformation by the two-plasmid excision detection system did not produce Ura⁺ colonies. However, 27 of 30 Leu⁺His⁺ colonies showed in PCR a shift in band size from ˜1.5 kb to ˜0.74 kb, characteristic of a deletion of eGFP. The lack of an Ura⁺ phenotype in the Leu⁺His⁺ colonies is probably due to interference of downstream gene expression by the ˜200 by res site. The deletion reaction was about ˜50% complete by day 7 as judged by the relative abundance of the excised to unexcised band. Representative recombinant plasmids were rescued into E. coli and subjected to by endonuclease digestions by AscI and SacI, and AscI and NheI, which revealed the new band sizes of 0.96 kb and 0.2 kb, respectively. Sequencing the ˜0.74 kb PCR product, as well as the product recombination site derived from pPbexc-Tn5053 confirmed that the recombination event occurred in a conservative manner.

Recombination Systems Where the Recombination Sites Are Non-Identical: phiC31, Bxb1, U153, and TP901-1.

PhiC31. The phiC31 integrase from Streptomyces bacteriophase phiC31 is a member of the large-serine resolvase subfamily. The 68 kDa protein catalyzes irreversible recombination between recombination sites attB and attP that are each ˜50 bp. PhiC31 was the first member of the large serine resolvase subfamily shown to be functional in a eukaryotic system (Groth et al., 2000; Thomason et. al., 2001). It catalyzes unidirectional and irreversible recombination in mammalian and plant cells (Groth et al., 2000; Ow et al., 2001; Belteki et al., 2003; Marillonnet et al., 2004; Lutz et al., 2004). Using the phiC31 recombination system as a positive control for the large serine resolvases, the PCR assay showed a distinct shift in band size from ˜1.5 kb to ˜0.74 kb consistent with site-specific excision of the eGFP ORF. Determining the PCR product corresponding to the excision event confirmed precise recombination between the intramolecular attP and attB sites. In the PCR assay, excision was found in 29 out of 30 Leu⁺His⁺ colonies. The relative intensities of the excised to unexcised bands suggested the deletion was ca. ˜80% complete 7 days after plating (See Table 1).

TP901-1. The temperate bacteriophage TP901-1 is from Lactococcus lactis subsp. cremoris 901-1. The phage can lysogenize the host by site-specific integration (Christiansen et al., 1994). The Tn901-1 integration system is localized to a 2.8 kb fragment of the phage genome which contains both a large-serine resolvase ORF and an attP site. The 485 aa TP901-1 resolvase is sufficient to integrate plasmids containing the attP site into an attB site in the host genome, whereas the excision of an integrated molecule requires an additional excisionase protein (Christiansen et al., 1996). Similar to that of the phiC31, the attP×attB reaction is uni-directional (Breuner et al., 1999). Recombination takes place within a common 5 by TCAAT core sequence shared by both attP and attB. The minimal sizes of the attP and attB sites appear to be 56 by and 43 bp, respectively (Breuner et al., 2001).

Unlike many of the systems described above, neither the attP site nor the attL (attP×attB hybrid) site interfered with expression of the downstream gene, as exemplified by expression of eGFP in pPB-TP901 prior to recombination, and ura4 after recombination. A corresponding shift in PCR product size from ˜1.5 kb to ˜0.74 kb was also detected in 99% of the analyzed colonies. Similar to phiC31, the relative band intensities indicated an approximate 80% completion 7 days after plating (See Table 1). The excision product was rescued into E. coli for cleavage by AscI and SacI, and AscI and NheI, which showed the characteristic bands of 0.96 kb and 0.2 kb. Precise recombination was found in the sequence of the ˜0.74 kb PCR product as well as the hybrid recombination site derived from pPBexc-TP901. During the course of this study, the TP901-1 was reported to function in mammalian cells (Stoll et al., 2002), which is consistent with the data described here.

Bxb1. The Bxb1 system is from the Mycobacterium smegmati bacteriophage Bxb1 that is morphologically similar to mycobacteriophages L5 and D29 and has 86 genes within its genome of ca. 50.5 kb (Mediavilla, et al., 2000). Lysogeny within the host is mediated by a recombinase belonging to the large serine resolvase subfamily. The Bxb1 resolvase is about 500 aa and the recombination sites attP and attB are 39 by and 34 bp, respectively.

The construct used in the excision assay, pPB-Bxb1, was found unable to express eGFP, suggesting that the 39 by attP site between the promoter and the downstream gene interfered with gene expression. Cotransformation with pPB-Bxb1 and pNMT-Bxb1 also failed to yield Ura⁺ colonies above the background rate. However, the PCR assay detected the ˜1.5 kb to ˜0.74 kb band shift in 99% of 30 Leu⁺His⁺ colonies. Hence, deletion without a corresponding Ura⁺ phenotype is most likely due to an attL (hybrid site of attP×attB) site interfering with gene expression when incorporated into the transcript leader region. Representative pPB-Bxb1 excision products were isolated from S. pombe for transformation into E. coli. AscI and SacI cleavage released a 0.96 kb band, and AscI and NheI cleavage released a 0.2 kb fragment. Precise site-specific recombination was confirmed from the DNA sequence of the ˜0.74 kb PCR product as well as the attL site derived from pPBexc-Bxb1. Based on the band intensities of the PCR assay, the deletion reaction was judged to be ˜95% complete by day 7 (See Table 1).

U153. Bacteriophage U153 (phiCU-SI153/95) is from the gram-positive, food-borne pathogen Listeria monocytogenes. It is capable of mediating transduction within a narrow host range (Hodgson, 2000). U153 has a genome size of 40.8 kb and encodes a large-serine recombinase (integrase) for lysogen formation. This 453 aa integrase, related to those of phiC31, TP901 and R4, recombines a 57 by recombination site, attP, with a 51 by recombination site, attB. The protein is sufficient to integrate plasmids containing attP site into the bacterial attB host target (Lauer et al., 2002).

Like the Bxb1 system, the pPB-U153 construct expressed eGFP, and the two-plasmid detection system yielded Ura⁺ colonies. This indicates that neither attP nor attL abolishes downstream gene expression. In PCR assays, the ˜1.5 kb to ˜0.74 kb band shift was detected in 28 of 30 colonies. Representative excision derivatives of pPB-U153, passed through E. coli, were cleaved with AscI and SacI, and with AscI and NheI. The appearance of the 0.96 kb and 0.2 kb fragments, respectively, confirmed site-specific deletion. DNA sequencing of the ˜0.74 kb PCR product as well as the hybrid recombination site derived from pPBexc-U153 confirmed conservative site-specific recombination. Based the relative intensities of the excised and unexcised bands in the PCR assay, this recombination system was the least efficient system among those tested, with only an estimated 35% of the DNA molecules having undergone recombination by day 7 (See Table 1).

Excision of Nuclear DNA. In a second assay for recombination, a series of plasmids, pRLPB-X, where X denotes the particular recombination system (FIG. 2A), were constructed with the same P_{NMT}-attP-eGFP-attB fragments as described in the pPB-X series (FIG. 1A). Downstream of attB lies a bsd (blasticidin resistance protein) coding region and a 1.8 kb ura4 fragment, but the construct lacks an ARS for autonomous replication. These constructs were linearized by cleavage with endonuclease Stu1 and transformed individually into S. pombe Sp223 (h⁻ leu1.32 ura4.294 ade.216). Uracil prototrophic colonies were examined by Southern blotting of BamHI cleaved DNA for homologous recombination of the introduced DNA into the ura4-294 mutant allele. The homologous insertion of a single copy of pRLPB-X into the ura4-294 locus produced a structure depicted in FIG. 2C, in which a ura4 DNA probe detected 10.2 kb and 3.4 kb BamHI fragments instead of the 6.8 kb band in Sp223. Representative single copy integrants of pRLPB-X were subsequent tested for site-specific recombination between the genomic recombination sites that flank eGFP. Recombinase was provided by secondary transformation with pNMT-X (FIG. 2D). Individual Lei⁺Ura⁺ colonies were assayed by PCR for conversion of a ˜1.6 kb band before recombination to a ˜0.80 kb band after recombination (the exact sizes of these PCR products differs slightly depending the length of the particular recombination site used in pRLPB-X).

Table 2 summarizes the data from the recombination systems tested which demonstrated site-specific excision of chromosomal DNA (eGFP) through site-specific recombination. Based on band intensities of the PCR products representing the excised and unexcised junctions, the ParA, Bxb1 and TP901 systems showed complete excision of eGFP in 7 day old colonies; CinH, phiC31, Tn1721 and Tn5053 showed ˜90% excision and U153 was the least efficient at ˜45% excision. With 14-day old colonies, however, the above recombination systems were close to complete excision. Genomic DNA of representative clones was also cleaved with BamH1 and probed with ³²P ura4 DNA. The ura4 probe detected the expected 10.2 kb and 3.4 kb bands before recombination, and the expected 9.2 kb and 3.4 kb bands after recombination. The change from a 10.2 to a 9.2 kb band is consistent with the loss of a ˜1 kb eGFP-att fragment. In cases where the colony represents a mixture of cells with complete and incomplete excision, a mix of both non-recombined and recombined genomes were found.

TABLE 2
Intramolecular chromosomal recombination in S. pombe

| | | | | | cfu with | |
|---|---|---|---|---|---|---|
| Recombi- | | | | Ura⁺ Leu⁺ | excision/ | Estimated |
| nation | Genomic | Transfected | Ura⁺ Leu⁺ | cfu/total | cfu | completion |
| System | construct | construct | cfu ^{a} | cfu (10⁻⁴) ^{a} | analyzed ^{b} | rate ^{c} |
| CinH | pRLPB-CinH | pNMT-CinH | 533 ± 214 | 1.5 ± 0.94 | 23/30 | 90% |
| parA | pRLPB-ParA | pNMT-ParA | 359 ± 302 | 0.81 ± 0.49 | 29/30 | 100% |
| Tn1721 | pRLPB-1721 | pNMT-Tn1721 | 301 ± 259 | 2.2 ± 1.9 | 25/27 | 90% |
| Tn5053 | pRLPB-5053 | pNMT-Tn5053 | 792 ± 769 | 2.3 ± 2.6 | 25/28 | 90% |
| phiC31 | pRLPB-C31 | pNMT-phiC31 | 735 ± 383 | 1.4 ± 1.1 | 23/29 | 90% |
| TP901-1 | pRLPB-TP | pNMT-TP901 | 2242 ± 360 | 3.7 ± 3.2 | 30/30 | 100% |
| Bxb1 | pRLPB-Bxb1 | pNMT-Bxb1 | 90 ± 60 | 0.89 ± 0.81 | 30/30 | 100% |
| U153 | pRLPB-U153 | pNMT-U153 | 1316 ± 332 | 20 ± 10 | 15/17 | 45% |

^{a} cfu = colony forming units; mean ± SD from 3 independent experiments.
^{b} Detection of excision by PCR. Data from 10 colonies analyzed for each of 3 independent experiments.
^{c} Estimate based on PCR band intensities.

DNA inversion assay. To test for DNA inversion, the set of pPB-X constructs (FIG. 1) were modified such that one of the recombination sites, the ura4 proximal site, is placed in the opposite orientation (FIG. 3). Site-specific recombination in this set of pPBi-X constructs, where X represents the recombination system, would be expected to invert the intervening eGFP fragment. As before, each pPBi-X plasmid was co-transformed into S. pombe FY527 along with pNMT-X. PCR was used to screen the Leu⁺His⁺ transformants for inversion of the eGFP intervening DNA. In the absence of inversion, the primer set shown in FIG. 3A, consisting of a first primer located within P_{NMT} and a second primer corresponding to the 5′end of eGFP will fail to amplify a product in pPBi-X. After site-specific inversion, however, this set of primers will amplify a product of ˜1.6 from the pPBi-X—derived ‘inverted construct’ (FIG. 3C).

To determine if the PCR reaction could detect an inversion event, a control plasmid was constructed with the eGFP fragment (plus transcription terminator) placed in the antisense orientation with respect to the P_{NMT} promoter (pNMTGFPrev). PCR using primers 1 and 3 generated a ˜1.6 kb band. The longer size PCR product is due to inclusion of the transcription terminator in the eGFP fragment (the exact size of the PCR also depends on the length of the recombination sites of the “X” recombination system). A second control was conducted using the Cre-lox site-specific recombinase system that is known to be capable of site-specific inversion in eukaryotic cells. The pPBi-Cre plasmid with a set of oppositely situated lox sites was transformed into S. pombe FY527 along with pNMT-Cre and Leu⁺His⁺ colonies were tested by PCR. As expected, the ˜1.6 kb band was found, but the inversion was only ˜50% complete. This is expected, as the Cre-mediated recombination reaction is reversible, with the equilibrium consisting of both inverted and non-inverted molecules.

The inversion tests with the pPBi-X plasmids showed site-specific inversion by the large serine resolvases Bxb1, phiC31, TP901, and U153. Although U153 indicated weaker inversion activity, this is consistent with its relatively lower activities for excision (above section) and integration (below section). Among the small serine resolvases, Tn1721 and Tn5053 were capable of causing DNA inversions, although at relatively low rates. The small serine resolvase family appears to be subdivided between the Tn3 and Tn21 subgroups, with CinH, Tn1721 and Tn5053 in the Tn21 subgroup and ParA in the Tn3 subgroup (Kholodii, 2001). Within the Tn21 subgroup, Tn5053 (Kholodii, 1995) and Tn1721 (Altenbuchner and Schmitt, 1983) have shown low inversion activity in bacteria, ˜10% the rates for excision. (Altenbuchner and Schmitt, 1983).

### Recombination Systems Capable of Inducing an Inter-Molecular Recombination Event

DNA Integration Assay. The data above show the ability of these recombination systems to perform an intra-molecular recombination event. To test for inter-molecular recombination, a first assay tests the cointegrate formation of two plasmids: the acceptor pHisB-X (FIG. 4A) and the donor pLeuP-X (FIG. 4B), where X indicates the recombination system. The acceptor construct pHisB-X contains one recombination site (such as attB), but lacking a promoter upstream of the ura4 ORF. It also contains his3 for selection, and an ARS for autonomous replication in S. pombe. The donor construct pLeuP-X contains the complementary recombination site (such as attP) inserted into a modified pNMT-TOPO vector that has leu for selection, but lacking ARS for replication. For S. pombe to become leucine autotrophic, pLeuP-X may be maintained in the cell by incorporating into a host chromosome, or integrating into the replication proficient plasmid pHisB-X.

To provide the recombinase for a possible attB×attP cointegration event, a third construct was cointroduced. This construct, derived from pNMT-X, was modified to remove both the leu selectable marker, and the ARS replication region. Hence, the modified construct, pNMTAS-X (FIG. 4C), is intended to provide transient expression of a recombinase gene. The three constructs were co-transformed into FY527, holding constant the plasmid concentrations of the acceptor and donor constructs (0.6 μg each) while varying the concentration of the recombinase construct (0.2 μg to 2 μg).

As control, the cointegration system was tested with the Cre-lox system, using both wild type and mutant (binding site) lox sites (Albert et al., 1995; Thomson et al., 2003). Under low Cre enzyme concentration, the mutant lox sites have a more unidirectional recombination reaction and hence can produce a more stable cointegrate molecule. Use of the Cre mediated integration system verified that the system functions as expected. Increasing amounts of the cre expression construct yielded higher numbers of leucine autotrophic S. pombe colonies. PCR was used to determine whether leucine autotrophy was due to plasmid-plasmid Cre-mediated integration. The PCR detection strategy was identical to that used for the two-plasmid excision test with one primer in the P_{NMT} promoter and the other primer in the ura4 ORF. For the Cre positive control experiment using the wild type, all of the colonies analyzed produced the predicted band size (Table 3).

TABLE 3
Intermolecular recombination in S. pombe

| | | | | | cfu with |
|---|---|---|---|---|---|
| | | | | | cointegration |
| Recombination | | | | His⁺ Leu⁺ | junction/cfu |
| system | Transfected constructs | | | cfu^{a} | analyzed^{b} |
| Cre | pHisB-Cre | pLeuP-Cre | pNMTAS-Cre | + | 19/19 |
| CinH | pHisB-CinH | pLeuP-CinH | pNMTAS-CinH | − | 0/0 |
| ParA | pHisB-ParA | pLeuP-ParA | pNMTAS-ParA | − | 0/0 |
| Tn1721 | pHisB-Tn1721 | pLeuP-Tn1721 | pNMTAS-Tn1721 | − | 0/0 |
| Tn5053 | pHisB-Tn5053 | pLeuP-Tn5053 | pNMTAS-Tn5053 | − | 0/0 |
| TP901-1 | pHisB-TP901 | pLeuP-TP901 | pNMTAS-TP901 | + | 19/19 |
| Bxb1 | pHisB-Bxb1 | pLeuP-Bxb1 | pNMTAS-Bxb1 | + | 19/19 |
| U153 | pHisB-U153 | pLeuP-U153 | pNMTAS-U153 | + | 6/19 |

^{a}cfu = colony forming units; − indicates ≦ background rate (without recombinase plasmid control); + indicates > background rate.
^{b}PCR detection of cointegrate junction. Data from 3 independent experiments where 5 to 7 colonies were analyzed.

In this cointegrate formation assay, each of the large serine recombinases, TP901-1, Bxb1, and U153, yielded Leu⁺ colonies that also corresponded with plasmid-plasmid cointegration generating a ˜0.74 kb PCR product (See Table 3). In Bxb1 and TP901-1, the ˜0.74 kb recombination junction was detected in every colony analyzed. In U153, however, it was detected in only 6 of 19 colonies. This suggests that the U153 system may be less efficiency in cointegration, as it is for the deletion reaction (See Table 1). Alternatively, it remains possible, that the U153 recombinase may be promoting the stabilization of the replication deficient leu plasmid, such as facilitating its integration into the host genome. For the small serine resolvases CinH, ParA, Tn1721, and Tn5053, each failed to yield Leu⁺ transformant over the background rate. This indicates a lack of cointegrate plasmid formation, or a highly unstable reaction that leads back to excision and followed by loss of the leu donor plasmid. The latter interpretation is not likely given that in the Cre-lox control experiments, the reversible wild type lox sites yielded Leu⁺ transformations.

Site-Specific Integration into the Host Genome. To test site-specific integration into nuclear DNA, a series of target sites was engineered into the S. pombe genome. This series of plasmids, pRLBZ-X, where “X” denotes the particular recombination system (FIG. 5A), comprise of a recombination site (such as attB) upstream of a bsd (blasticidin resistance) coding region devoid of its promoter, followed downstream by a 1.8 kb ura4⁺ DNA fragment. This construct was made linear through cleavage by endonuclease Stul and transformed into Sp223. Uracil prototrophic colonies that integrated a single copy of pRLBZ-X by homologous recombination into the ura4-294 mutant locus were identified by molecular analysis and selected as target lines (FIG. 5C). In Southern blots of BamHI cleaved genomic DNA, these colonies show a ˜11.2 kb band when hybridized to ³²P labeled ura4 DNA, in contrast to the 6.8 kb band found in Sp223. These target lines were then transformed with pLeuP-X and pNMTAS-X (FIG. 5D). Recombinase produced by pNMTAS-X promoted site-specific integration of pLeuP-X into the genomically situated pRLBZ-X, leading to the genomic structure depicted in FIG. 5E.

Table 4 shows the data of representative recombination systems. Individual Leu⁺Ura⁺ colonies were analyzed by PCR for the presence of a ˜0.80 kb band indicative of the joining between the bsd and P_{NMT} DNA (the exact size of the PCR product depends on the length of the recombination site used). Site-specific integration of pLeuP-X into the pRLBZ-X genomic construct was detected by PCR in all of the Ura⁺Leu⁺ colonies derived from recombination systems phiC31, TP901-1 and Bxb1.

TABLE 4
Site-specific integration into the S. pombe chromosome.

| Recombi- | | | | Ura⁺ | Ura⁺ Leu⁺ | |
|---|---|---|---|---|---|---|
| nation | Genomic | | | Leu⁺ | cfu/total | Ura⁺ Leu⁺ |
| system | construct | Transfected constructs | | (10²) ^{a} | cfu (10⁻⁴) ^{a} | cfu |
| phiC31 | pPLBZ-phiC31 | pLeuP-phiC31 | pNMTAS-phiC31 | 0.72 | 0.268 | 29/30 |
| TP901-1 | pRLBZ-TP901 | pLeuP-TP901 | pNMTAS-TP901 | 2.63 | 0.535 | 26/30 |
| Bxb1 | pRLBZ-Bxb1 | pLeuP-Bxb1 | pNMTAS-Bxb1 | 3.91 | 0.636 | 24/30 |

^{a} cfu = colony forming units; − indicates ≦ background rate (without recombinase plasmid control); + indicates > background rate.
^{b} PCR detection of cointegrate junction. Data from 3 independent experiments where 8 colonies were analyzed. Results were confirmed by S. blot.

Genomic DNA from representative integrant colonies was also cleaved with endonuclease BamH1 and hybridized to ³²P labeled ura4 DNA. The wild type ura4 locus shows a band of about ˜6 kb (FIG. 5C). The homologous insertion of pRLBZ-X increases the size of the BamHI fragment to ˜11.2 kb (FIG. 5D), and the recombinase-mediated integration of pLeuP-X further increases the size of the BamHI fragment to ˜16.2 kb (FIG. 5E).

Site-specific recombination in plant cells test for functional site-specific recombination in plants, the generic plasmid pN6PB-X was used, where “X” denotes the particular recombination system. It comprises of a fragment from pPB-X inserted into a pCambia1301 Agrobacterium vector backbone. The pPB-X fragment consisting of a set of recombination sites flanking an eGFP coding region (FIG. 6A). In pN6PB-X, the fragment is situated between the CaMV 35S RNA promoter (35S) and a beta-glucoronidase gene (gus) coding region, followed by a hygromycin resistance gene (hpt) for plant selection. A corresponding pCK-X (FIG. 6C), where X specifies the recombination system, comprises of a recombinase gene under the control the CaMV 35S RNA promoter within an Agrobacterium vector backbone.

In a first experiment, Arabidopsis or tobacco protoplasts were prepared from young leaves and electroporated in presence of DNA as described previously (Dale and Ow, 1990; Morgan and Ow, 1995). The protoplasts were transfected with pN6PB-X, pCK-X, or both, where X specifies the recombination system. To enhance the probability of cells that take up pN6PB-X would also take up pCK-X, the two plasmids were transfected at a molar ratio of 1 pN6PB-X to 10 pCK-X (FIG. 6A, B). Transfected cells were incubated for 2 to 3 days prior to DNA extraction. The extracted DNA was then subjected to PCR detection of a ˜0.5 kb band representing the recombination junction (exact size of PCR depends on length of recombination site used).

As an example, when pN6PB-Bxb1 was transfected into Arabidopsis protoplasts in the absence of pCK-Bxb1, the PCR reaction yielded a ˜1.5 kb band representing the span of DNA that includes the eGFP fragment (FIG. 6A). When pN6PB-Bxb1 was co-transfected with pCK-Bxb1, however, a new ˜0.5 kb band was found, which is consistent with excision of eGFP. This 0.5 kb band was not found when either one of the two plasmids were omitted. In another example, a time-dependent recombination reaction was observed. When pN6PB-ParA was transfected into tobacco protoplasts in the absence of pCK-ParA, the PCR reaction yielded a ˜1.5 kb band, as well as a background band of 0.25 kb that is also found with tobacco DNA in the absence of transfected plasmids. When pCK-ParA was included with pN6PB-ParA, however, a new ˜0.5 kb band representing the excision junction was detected. For protoplasts incubated for 2 days, both the ˜1.5 and the ˜0.5 kb bands were amplified. For protoplasts incubated for 3 days, however, the ˜0.5 kb band became more intense, while the ˜1.5 kb band was no longer detected. This shift from the ˜1.5 kb to the ˜0.5 kb band suggests that excision had reached near 100% completion.

In another example, transgenic Arabidopsis plants were generated by Agrobacterium transformation with GV3101 harboring pN6PB-X . Hygromycin resistant plants were analyzed by PCR to contain the pN6PB-X derived T-DNA, the ˜1.5 kb band as depicted in FIG. 6B. Four N6PB-X plants, from each of the “X” recombinase system, were transformed a second time, with pCK-X (FIG. 6C), which confers kanamycin resistance and expresses the “X” recombinase gene. Small seedlings resistant to kanamycin were screen by PCR for recombinase-mediated excision of eGFP as indicated by the ˜0.5 kb PCR product depicted in FIG. 6D. As an example, a ˜0.5 kb band was amplified from the DNA of the pN6PB-ParA transformed seedlings, which indicates site-specific excision of the intervening eGFP fragment. The ˜1.5 kb band, however, was also amplified along with the ˜0.5 kb band, hence the recombination reaction was not yet complete at this early stage of plant development. In the case of pN6PB-ParA, excision of eGFP also led to the expression of the gus gene, as revealed by the blue staining of leave cells that is characteristic of GUS enzyme activity. Together, these data are consistent with site-specific recombination in the plant genome.

In site-specific integration, aside from having the DNA placed into a known location, an additional advantage is that recombinase-mediated integration can be more efficient than the host-mediated random insertion of DNA (Albert et al., 1995; Day et al., 2000; Srivastava and Ow, 2002; Belteki et al., 2003; Srivastava et al., 2004; Lutz et al., 2004). An example of this higher rate of transformation is illustrated by the following experiment. Transgenic tobacco plants were generated by Agrobacterium-mediated transformation with pYMP72 (FIG. 7A), which comprises of a pCambia2300 vector backbone (Cambia, Canberra), and the following genes within the T-DNA left (LB) and right (RB) borders: npt (kanamycin resistance gene), gus (beta-glucuronidase gene), and a set of attP sites comprising of the attP sites of Bxb1, U153, TP901-1 and phiC31. Plant lines harboring a single copy of the transgene were screened by Southern blotting on XbaI cleaved DNA, which cuts pYWP72 at two sites flanking the promoter that drives gus (FIG. 7B). Single target copy lines were defined as those that showed a single hybridizing band to either nptII or gus DNA probe, as well as show proficient expression of gus. The single-copy target lines were then used for a second transformation with pYWSB2 in the presence or absence of a co-transfected pCK-X or pCK-Xn plasmid that expresses the “X” recombinase (FIG. 7C). In the pCK-Xn version, the recombinase is fused to a nuclear localization signal to facilitate nuclear entry. The pYWSB2 construct contains hpt (hygromycin resistance gene) and two sets of corresponding attB sites (each set comprising of the attB sites of Bxb1, U153, TP901-1 and phiC31) flanking luc (luciferase gene). The “X” recombinase is expected to promote the recombination between the “X” attP at the pYWP72 transgenic locus and either of the “X” attB sites in pYWSB2. Recombination of the luc-upstream attB would yield the structure shown in FIG. 7D, whereas recombination of the luc-downstream attB would yield the structure shown in FIG. 7E.

Leaf explants from the four single copy pYMP72-transgenic lines were subjected to direct DNA transformation (particle bombardment) followed by incubation of the leaf explants in hygromycin-containing medium. When functional recombinase-expressing DNA was not included in the transformation by pYWSB2, <2% of leaf explants formed calli. Since the hpt fragment contains a promoter, the low frequency of transformation is likely the random integration of pYWSB2. In contrast, when pCK-Bxb1, pCK-Bxb1n or pCK-TP901n was included in the transformation, between 10 to 71% of the leaf explants (depending on the cell line) showed callus formation along with regenerating shoots. This is comparable to the data obtained with the control pCK-phiC3ln DNA. This indicates that recombinase-mediated transformation is more efficient than host-mediated random insertion of the introduced DNA.

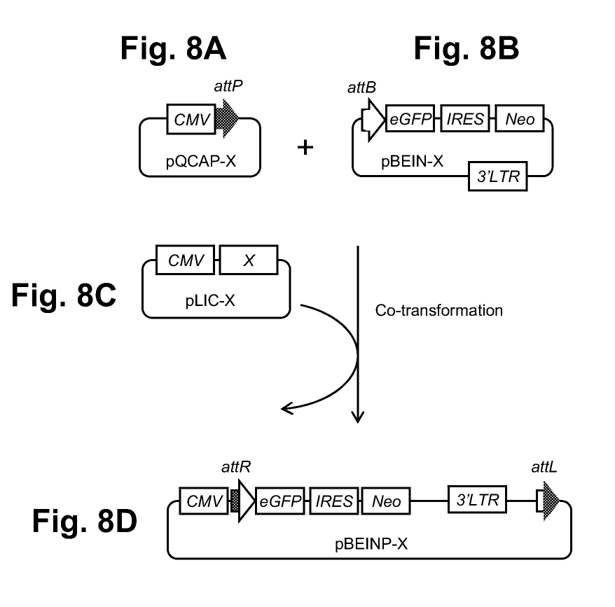

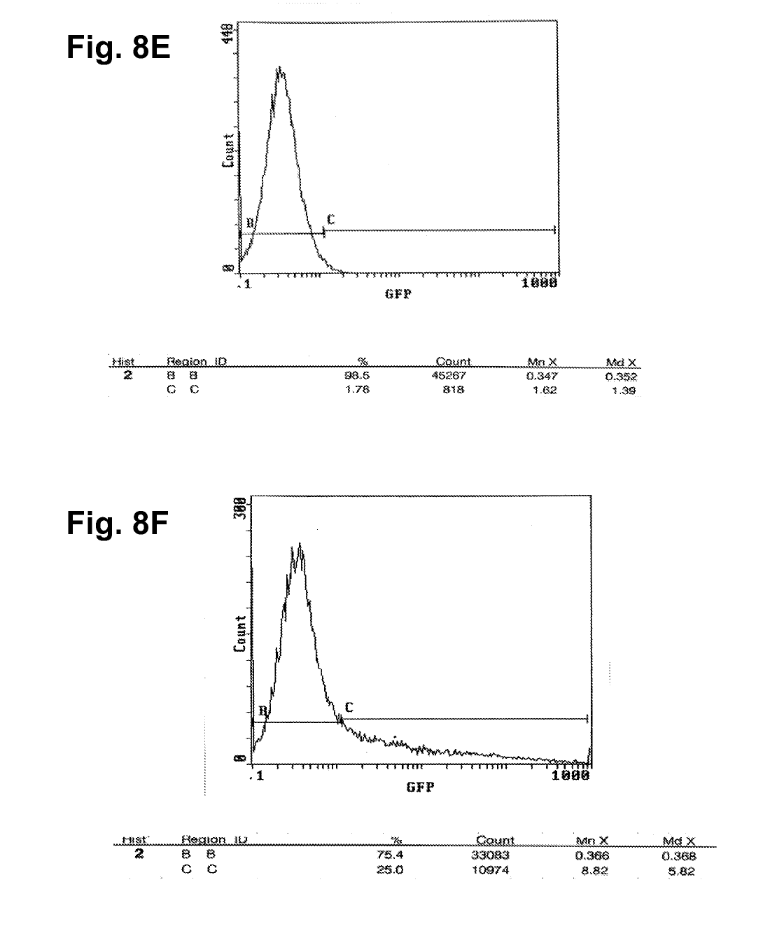

Site-specific recombination in animal cells. To test site-specific recombination in mammalian cells, one assay relied on the site-specific recombination between recombination sites situated on two separate DNA molecules. A first plasmid, pQCAP-X (FIG. 8A), where X indicates the recombination system, comprises of a CMV promoter upstream of an attP site; a second plasmid pBEIN-X (FIG. 8B), where X indicates the recombination system, comprises of an attB upstream of a promoterless eGFP; and a third plasmid pLIC-X (FIG. 8C), where X indicates the recombination system, comprises of the corresponding recombinase gene expressed by a CMV. Site-specific recombination between attB and attP would fuse the CMV promoter to eGFP (FIG. 8D). Expression of eGFP would be expected if the hybrid site does not interfere with expression of the downstream gene (and this may differ depending on the translational efficiency in a particular cell type). FIGS. 8E and 8F shows an example of the recombination observed with the Bxb1 system. When examined by microscopy, florescent cells, indicative of eGFP expression, were not found when transfected without DNA, or with pBEIN-Bxb1 and pQCAP-Bxb1, but without pLIC-Bxb1. However, when all three plasmids were included in the transfection, faintly florenscent cells were detected. A flourescent flow sorter was to quantify the number of eGFP expressing cells. As shown in FIG. 8F, 20 to 25% of the cell population expressed eGFP, when all three plasmids were used in the transfection, while few fluorescent cells were found when transfected without DNA, or with pBEIN-Bxb1 and pQCAP-Bxb1, but without pLIC-Bxb1 (FIG. 8E). This expression, which depended on the presence of the Bxb1 recombinase-expressing construct, indicates Bxb1-catalyzed site-specific recombination in the mammalian cell line.

Specificity Assay. In the use of site-specific recombination systems for genome engineering, a future trend will be toward the deployment of several different recombination systems within a transgenic cell. To address whether the recombinases might cross react with related recombination sites, the deletion assay described in FIG. 1 was tested with heterologous recombinases. Within the small serine resolvase subfamily, when a pPB-X construct was tested with a heterologous pNMT-X construct, site-specific deletion was not observed (See Table 5). The same held true for the recombinases from the large serine resolvase subfamily (See Table 6). This indicates that the recombinase/recombination site specificity is maintained in a eukaryotic cell.

TABLE 5
Specificity of recombination of the small serine resolvase subfamily

Recombination sites^{b}

| Recombinase^{a} | phiC31 | TP901-1 | Bxb1 | U153 |
|---|---|---|---|---|
| phiC31 | Y | N | N | N |
| TP901-1 | N | Y | N | N |
| Bxb1 | N | N | Y | N |
| U153 | N | N | N | Y |

^{a}pNMT-X constructs, where X indicates the recombinase.
^{b}pPB-X constructs, where X indicates the recombination system.
Y = excision detected by PCR;
N = excision not detected.

TABLE 6
Specificity of recombination of the large serine resolvase subfamily

Recombination sites^{b}

| Recombinase^{a} | CinH | ParA | Tn1721 | Tn5053 |
|---|---|---|---|---|
| CinH | Y | N | N | N |
| ParA | N | Y | N | N |
| Tn1721 | N | N | Y | N |
| Tn5053 | N | N | N | Y |

^{a}pNMT-X constructs, where X indicates the recombinase.
^{b}pPB-X constructs, where X indicates the recombination system.
Y = excision detected by PCR;
N = excision not detected.

### REFERENCES

ALBERT H, Dale E C, Lee E, Ow D W. (1995) Site-specific integration of DNA into wild-type and mutant lox sites placed in the plant genome. Plant J. 7, 649-59.
ALTENBUCHNER J, Schmitt R. (1983) Transposon Tn1721: Site-Specific Recombination

Generates Deletions and Inversion. Mol. Gen. Genet. 190, 300-308.

BELTEKI, G., Gertsenstein, M., Ow, D. W., Nagy, A. (2003) Site-specific cassette exchange and germline transmission with mouse ES cells expressing φC31 integrase. Nature Biotechnology 21, 321-324.
BREUNER A, Brondsted L, Hammer K. (1999) Novel organization of genes involved in prophage excision identified in the temperate lactococcal bacteriophage TP901-1. J Bacteriol. 181, 7291-7297.

BREUNER A, Brondsted L, Hammer K. (2001) Resolvase-like recombination performed by the TP901-1 integrase. Microbiology. 147, 2051-2063.
BURKARDT H J, Mattes R, Schmid K, Schmitt R. (1978) Properties of two conjugative plasmids mediating tetracycline resistance, raffinose catabolism and hydrogen sulfide production in Escherichia coli. Mol Gen Genet. 166, 75-84.
CHRISTIANSEN B, Brondsted L, Vogensen FK, Hammer K. (1996) A resolvase-like protein is required for the site-specific integration of the temperate lactococcal bacteriophage TP901-1. J Bacteriol. 178, 5164-5173.
CHRISTIANSEN B, Johnsen MG, Stenby E, Vogensen F K, Hammer K. (1994) Characterization of the lactococcal temperate phage TP901-1 and its site-specific integration. J Bacteriol. 176, 1069-76.
DALE E C and Ow D W. (1990) Intra- and intermolecular site-specific recombination in plant cells mediated by bacteriophage P1 recombinase. Gene 91, 79-85.

DALE E C and Ow D W. (1991) Gene transfer with subsequent removal of the selection gene from the host genome. Proc. Natl. Acad. Sci. USA 88, 10558-10562.
DAY C D, Lee E, Kobayashi J, Holappa L D, Albert H, Ow D W. (2000) Transgene integration into the same chromosome location can produce alleles that express at a predictable level, or alleles that are differentially silenced. Genes Dev. 14 , 2869-2880.
FERREIRA L P, Lemos E G, Lemos M V. (2002) Transposon Tn1721 distribution among strains of Xylella fastidiosa. FEMS Microbiol Lett. 208, 163-168.
FISHER, D K and Guiltinan M J. (1995) Rapid, Efficient Production of Homozygous Transgenic Tobacco Plants with Agrobacterium tumefiaciens: A Seed to Seed protocol. Plant Molec. Biol. Rep. 13, 278-289.
GERLITZ M, Hrabak O, Schwab H. (1990) Partitioning of broad-host-range plasmid RP4 is a complex system involving site-specific recombination. J Bacteriol. 172, 6194-203.
GIETZ, R. D. and R. A. Woods. (2002) Transformation of yeast by the LiAc/SS carrier DNA/PGE method. Methods in Enzymology 350, 87-96.
GILBERTSON L, Dioh W, Addae P, Ekena J, Keithly G, Neuman M, Peschke V, Petersen M, Samuelson C, Subbarao S, Wei L, Zhang W, Barton K. (2003) Cre/lox mediated marker gene excision in transgenic crop plants. In: Plant Biotechnology 2002 and Beyond (ed. IK Vasil), Kluwer Academic Publishers, Dordrecht, The Netherlands, pp. 225-228.
GROTH A C, Olivares E C, Thyagarajan B, Calos M P. (2000) A phage integrase directs efficient site-specific integration in human cells. Proc Natl Acad Sci USA. 97, 5995-6000.
HERITAGE J. (2004) The fate of trangenes in the human gut. Nat. Biotech. 22, 170-172.
HODGSON D A. (2000) Generalized transduction of serotype 1/2 and serotype 4b strains of Listeria monocytogenes. Mol Microbiol. 35, 312-323.
KHOLODII G Y. (1995) Inversion Activity of the Tn5053 and Tn402 Resolution System, Which Possess and Uncommon res Region. Russ. J. Genetics. 31, 1698-1703.
KHOLODII G. (2001) The shuffling function of resolvases. Gene 269, 121-130.
KHOLODII G Y, Mindlin S Z, Bass I A, Yurieva O V, Minakhina S V, Nikiforov V G. (1995) Four genes, two ends, and a res region are involved in transposition of Tn5053: a paradigm for a novel family of transposons carrying either a mer operon or an integron. Mol Microbiol. 17, 1189-2000.
KHOLODII G Y, Yurieva O V, Lomovskaya O L, Gorlenko Z, Mindlin S Z, Nikiforov V G. (1993) Tn5053, a mercury resistance transposon with integron's ends. J Mol Biol. 230, 1103-1107.
KRASNOW M A, Cozzarelli N R. (1983) Site-specific relaxation and recombination by the Tn3 resolvase: recognition of the DNA path between oriented res sites. Cell 32, 1313-1324.
LAUER P, Chow M Y, Loessner M J, Portnoy D A, Calendar R. (2002) Construction, characterization, and use of two Listeria monocytogenes site-specific phage integration vectors. J Bacteriol. 184, 4177-4186.
LUTZ K A, Corneille S, Azhagin A K, Svab Z, Maliga P. (2004) A novel approach to plastid transformation utilizes the phiC31 phage integrase. Plant J. 37, 906-913.
MARILLONNET, S., Giritch, A., Gils, M., Kandzia, Klimyuk, V., Gleba, Y. (2004) In planta engineering of viral RNA replicons: Efficient assembly by recombination of DNA modules delivered by Agrobacterium. Proc. Natl. Acad. Sci. U.S.A. 18, 6852-6857.
MEDIAVILLA J, Jain S, Kriakov J, Ford M E, Duda R L, Jacobs W R Jr, Hendrix R W, Hatfull G F. (2000) Genome organization and characterization of mycobacteriophage Bxb1. Mol Microbiol. 38, 955-970.
MORGAN, M. K., Ow, D. W. (1995) Polyethylene glycol-mediated transformation of tobacco leaf mesophyll protoplasts: an experiment in the study of Cre-lox recombination. In: Methods In Plant Molecular Biology, A Laboratory Manual (Maliga, P., Klessig, D., Cashmore, A., Gruissem W., Varner, J., eds.), Cold Spring Harbor Press, pp. 1-17.
ORTIZ, D. F., Kreppel, L., Speiser, D. M., Scheel, G., McDonald, G., Ow, D. W. (1992) Heavy metal tolerance in the fission yeast requires an ATP binding cassette-type vacuolar membrane transporter. EMBO J 11: 3491-3499.
OW D W. (2002) Recombinase-directed plant transformation for the post genomic era. Plant Molec. Biol. 48, 183-200.
OW, D. W., Calendar, R., Thomason, L. (2001) DNA recombination in eucaryotic cells by the bacteriophage phiC31 recombination system, International (WIPO PCT) publication WO 01/07572, Feb. 1, 2001.
REED R R. (1981) Resolution of cointegrates between transposons gamma delta and Tn3 defines the recombination site. Proc Natl Acad Sci USA. 78, 3428-3432.
ROBERTS RC, Strom A R, Helinski D R. (1994) The parDE operon of the broad-host-range plasmid RK2 specifies growth inhibition associated with plasmid loss. J Mol Biol. 237, 35-51.
ROGOWSKY P, Schmitt R. (1985) Tn1721-encoded resolvase: structure of the tnpR gene and its in vitro functions. Mol Gen Genet. 200, 176-181.
SCHMITT R, Bernhard E, Mattes R. (1979) Characterisation of Tn1721, a new transposon containing tetracycline resistance genes capable of amplification. Mol Gen Genet. 172, 53-65.
SIMMONS N L. (1990) A cultured human renal epithelioid cell line responsive to vasoactive intestinal peptide. Exp Physiol. 75, 309-19.
SOBECKY P A, Easter C L, Bear P D, Helinski D R. (1996) Characterization of the stable maintenance properties of the par region of broad-host-range plasmid RK2. J Bacteriol. 178, 2086-2093.
SRIVASTAVA V and Ow D W. (2002) Biolistic mediated site-specific integration in rice. Molecular Breeding 8, 345-350.
SRIVASTAVA, V., Ariza-Nieto, M. and Wilson, A. J. 2004. Cre-mediated site-specific gene integration for consistent transgene expression in rice. Plant Biotech. J. 2, 169-179.
STOLL S, Ginsburg D S, Calos M P. (2002) Phage TP901-1 site-specific integrase functions in human cells. J Bacteriol. 184, 3657-3663.
THOMASON L C, Calendar R, Ow D W. (2001) Gene insertion and replacement in Schizosaccharomyces pombe mediated by the Streptomyces bacteriophage phiC31 site-specific recombination system. Mol Genet Genomics. 265, 1031-1038.
THOMSON J G, Rucker E B 3rd, Piedrahita J A. (2003) Mutational analysis of loxP sites for efficient Cre-mediated insertion into genomic DNA. Genesis 36, 162-167.
WATSON M A, Boocock M R, Stark W M. (1996) Rate and selectively of synapsis of res recombination sites by Tn3 resolvase. J Mol Biol. 257, 317-329

What is claimed is:

1. A method for obtaining site-specific recombination in a eukaryotic cell, the method comprising:
a. providing the eukaryotic cell
wherein
the eukaryotic cell comprises a polynucleotide that encodes a prokaryotic Bxb1 recombinase polypeptide,
wherein
the eukaryotic cell comprises a first site-specific recombination site and a second site-specific recombination site, wherein the first site-specific recombination site is a substrate for recombination with the second site-specific recombination site; and
b. contacting the first site-specific recombination site and second site-specific recombination site with a prokaryotic Bxb1 recombinase polypeptide, resulting in recombination between the first site-specific recombination site and second site-specific recombination site. The method of claim 1, wherein the eukaryotic cell is a plant cell.

2. The method of claim 1, wherein the eukaryotic cell is a yeast cell.

3. The method of claim 1, wherein the eukaryotic cell is an animal cell.

4. The method of claim 1, wherein the polynucleotide that encodes the prokaryotic Bxb1 recombinase polypeptide comprises SEQ ID NO:1.

5. The method of claim 1, wherein the polynucleotide that encodes the prokaryotic Bxb1 recombinase polypeptide encodes a recombinase polypeptide comprising SEQ ID NO:2.

6. The method of claim 1, wherein the prokaryotic Bxb1 recombinase polypeptide causes a site-specific excision of DNA,
wherein
the excision of the DNA is a result of recombination between the first site-specific recombination site and the second site-specific recombination site, and
wherein
the DNA that is excised is located between the first site-specific recombination site and the second site-specific recombination site on the same DNA molecule.

7. The method of claim 1, wherein the prokaryotic Bxb1 recombinase polypeptide causes a site-specific inversion of DNA
wherein
the inversion of the DNA is a result of recombination between the first site-specific recombination site and the second site-specific recombination site, and
wherein
the DNA that is inverted is located between the first site-specific recombination site and the second site-specific recombination site on the same DNA molecule.

8. The method of claim 1, wherein the prokaryotic Bxb1 recombinase polypeptide causes a site-specific integration of DNA
wherein
the integration of the DNA is a result of recombination between the first site-specific recombination site and the second site-specific recombination site, and
wherein
the DNA that is integrated is located between the first site-specific recombination site and the second site-specific recombination site on the same DNA molecule.

9. A method for obtaining site-specific recombination in a eukaryotic cell, the method comprising:
a. providing the eukaryotic cell
wherein
the eukaryotic cell comprises a polynucleotide that encodes a prokaryotic U153 recombinase polypeptide,
wherein
the eukaryotic cell comprises a first site-specific recombination site and a second site-specific recombination site, wherein the first site-specific recombination site is a substrate for recombination with the second site-specific recombination site; and
b. contacting the first site-specific recombination site and second site-specific recombination site with a prokaryotic U153 recombinase polypeptide, resulting in recombination between the first site-specific recombination site and second site-specific recombination sites.

10. The method of claim 9, wherein the eukaryotic cell is a plant cell.

11. The method of claim 9, wherein the eukaryotic cell is a yeast cell.

12. The method of claim 9, wherein the eukaryotic cell is an animal cell.

13. The method of claim 9, wherein the polynucleotide that encodes the prokaryotic U153 recombinase polypeptide comprises SEQ ID NO:19.

14. The method of claim 9, wherein the polynucleotide that encodes the prokaryotic U153 recombinase polypeptide encodes a recombinase polypeptide comprising SEQ ID NO:20.

15. The method of claim 9, wherein the prokaryotic U153 recombinase polypeptide causes a site-specific excision of DNA,
wherein
the excision of the DNA is a result of recombination between the first site-specific recombination site and the second site-specific recombination site, and
wherein
the DNA that is excised is located between the first site-specific recombination site and the second site-specific recombination site on the same DNA molecule.

16. The method of claim 9, wherein the prokaryotic U153 recombinase polypeptide causes a site-specific inversion of DNA,
wherein
the inversion of the DNA is a result of recombination between the first site-specific recombination site and the second site-specific recombination site, and
wherein
the DNA that is inverted is located between the first site-specific recombination site and the second site-specific recombination site on the same DNA molecule.

17. The method of claim 9, wherein the prokaryotic U153 recombinase polypeptide causes a site-specific integration of DNA,
wherein
the integration of the DNA is a result of recombination between the first site-specific recombination site and the second site-specific recombination site, and
wherein
the DNA that is integrated is located between the first site-specific recombination site and the second site-specific recombination site on the same DNA molecule.

18. A method for obtaining a eukaryotic cell having a stably integrated transgene, the method comprising:
a. introducing a first nucleic acid into a eukaryotic cell that comprises a first recombination site,
b. introducing a second nucleic acid comprising a transgene and a second recombination site, which second recombination site can serve as a substrate for recombination with the first recombination site; and
c. contacting the first and the second recombination sites with a recombinase polypeptide, wherein the recombinase polypeptide catalyzes recombination between first and second recombination sites, resulting in integration of the second nucleic acid at the first recombination site(s), thereby forming two hybrid sites flanking the inserted DNA.

19. The method of claim 18, wherein the recombinase polypeptide can mediate site-specific recombination between the first and second recombination sites, but cannot mediate recombination between the hybrid sites in the absence of an additional factor that is not present in the eukaryotic cell.

20. The method of claim 18, wherein the recombination sites and recombinase polynucleotide are selected from the group consisting of a bacteriophage Bxb1 recombination system, and a bacteriophage U153 recombination system.
